(19) United States
(12) Patent Application Publication
(10) Pub. No.: US 2024/0150709 A1
(43) Pub. Date: May 9, 2024

(54) COMPOSITIONS AND METHODS FOR MAKING PARASYMPATHETIC NEURONS

(71) Applicant: University of Georgia Research Foundation, Inc., Athens, GA (US)

(72) Inventors: Nadja Zeltner, Athens, GA (US); Hsueh Fu Wu, Athens, GA (US)

(21) Appl. No.: 18/503,100

(22) Filed: Nov. 6, 2023

Related U.S. Application Data
(60) Provisional application No. 63/422,777, filed on Nov. 4, 2022.

(51) Int. Cl. C12N 5/0793

(52) U.S. Cl. CPC C12N 5/0619; C12N 2500/32; C12N 2501/115; C12N 2501/13; C12N 2501/155; C12N 2506/00; C12N 2513/00; C12N 2533/52

(57) ABSTRACT

Parasympathetic neurons (parasymN) are important for unconscious body responses, including rest-and-digest and calming the body. Disclosed herein is a chemically defined differentiation protocol that generates parasympathetic neurons from stem cells. The protocol yields high efficiency and purity cultures that are electrically active and respond to specific stimuli. Their molecular characteristics and maturation stage are described and evidence for their use as a model for studying parasymN function and dysfunction. Cell populations and compositions formed from the resulting cells, as well as methods of their use for disease treatment, drug screening, and modeling of human disorders affecting parasymN are also provided.

### CROSS-REFERENCE TO RELATED APPLICATIONS

This application claims the benefit of and priority to U.S. Provisional Application No. 63/422,777, filed Nov. 4, 2022, which is hereby incorporated herein by reference in its entirety.

### STATEMENT REGARDING FEDERALLY SPONSORED RESEARCH

This invention was made with government support under grant number R01NS114567 awarded by the NIH. The government has certain rights in the invention. (37 CFR 401.14 f (4)).

### REFERENCE TO SEQUENCE LISTING

The Sequence Listing submitted as a text file named “UGA 2022-118-02 US.xml” created on Nov. 6, 2023, and having a size of 59,247 bytes is hereby incorporated by reference pursuant to 37 C.F.R. § 1.52(e)(5).

### FIELD OF THE INVENTION

The field of the invention generally relates to compositions and methods of differentiating stem cells into parasympathetic neurons.

### BACKGROUND OF THE INVENTION

The autonomic nervous system (ANS) of the periphery regulates body homeostasis and involuntary responses, such as heart rate and blood pressure. The two major components of the ANS are the sympathetic nervous system (SNS) and the parasympathetic nervous system (PSNS). While postganglionic sympathetic neurons (symNs) release norepinephrine to stimulate the fight-or-flight response, which causes increased heartbeat and vasocontraction, postganglionic parasympathetic neurons (parasymNs) release acetylcholine (ACh) to trigger the opposite response called “rest-and-digest” (Wehrwein, E. A., Orer, H. S. & Barman, S. M. Compr Physiol 6, 1239-1278 (2016)). Autonomic dysfunction has been found in various diseases, such as Parkinson's disease, Alzheimer's disease, hypertension, multiple system atrophy, diabetic autonomic neuropathy, spinal cord injury, COVID-19, and familial dysautonomia (FD) (Porzionato, A., et al. FEBS J 287, 3681-3688 (2020); Brook, R. D. & Julius, S. Am J Hypertens 13, 112S-122S (2000); Del Rio, R., Marcus, N. J. & Inestrosa, N.C. Front Physiol 11, 561749 (2020)). The “imbalance” of the ANS, meaning either the increased sympathetic or decreased parasympathetic tones, or both, are contributing to the autonomic neuropathy in these diseases (Yang, A., Liu, B. & Inoue, T. Eur J Neurosci 55, 1645-1657 (2022)).

Most clinical assessments of ANS function are indirect methods, for example skin conductance measurements indicating in/decreased SNS/PSNS activity (Zygmunt, A. & Stanczyk, J. Arch Med Sci 6, 11-18 (2010); Macefield, V. G. Clin Auton Res 31, 59-75 (2021)). Additionally, it is difficult to observe ANS specific responses without the interference from the central nervous system (CNS) and it is near impossible to extract primary human tissue for research. Human pluripotent stem cells (hPSC) technology provides an ideal platform to study function and dysfunction of human ANS neurons, on the premise that a defined neuron differentiation strategy is available (Saito-Diaz, K. & Zeltner, N. Clin Auton Res 29, 367-384 (2019); Zeltner, N. & Studer, L. Curr Opin Cell Biol 37, 102-110 (2015)).

While there are several published differentiation protocols to derive human symNs (Wu, H. F. & Zeltner, N. J Vis Exp (2020); Frith, T. J. R. & Tsakiridis, A. Curr Protoc Stem Cell Biol 49, e81 (2019); Kirino, K., Nakahata, T., Taguchi, T. & Saito, M. K. Sci Rep 8, 12865 (2018)), only one parasymN protocol was described (Takayama, Y., et al. Sci Rep 10, 9464 (2020)). According to current knowledge, parasymNs develop from Schwann cell precursors (SCPs), which are derived from the neural crest cell (NC) (Dyachuk, V., et al. Science 345, 82-87 (2014); Espinosa-Medina, I., et al. Science 345, 87-90 (2014)). This feature makes parasymNs developmentally different from symNs, which are differentiated directly from the NC (Ernsberger, U. & Rohrer, H. Neural Dev 13, 20 (2018)). Takayama et al., derived both symNs and parasymNs directly from autonomic progenitor cells, which does not reproduce the proper developmental process (Takayama, Y., et al. Sci Rep 10, 9464 (2020)). Furthermore, parasymNs and symNs are located in distinct ganglia, that are located relatively far away from each other. In the Takayama study the two neuron types emerge in the same clusters. Lastly, it is known that some symNs are cholinergic, normally the typical parasymN neurotransmitter, and that adrenergic symNs can switch to becoming cholinergic upon innervation of a tissue that is not innervated by parasymNs (Yang, B., Slonimsky, J. D. & Birren, S. J. Nat Neurosci 5, 539-545 (2002)). Thus, it is likely that their parasymN-like cells may be cholinergic symNs instead.

Thus, there remains a need for improved means of preparing parasympathetic neurons.

It is an object of the invention to provide compositions and methods for preparing parasympathetic neurons.

It is also an object of the invention to provide parasympathetic neurons and methods of use thereof for disease modeling, drug screening, and transplantation therapy.

### SUMMARY OF THE INVENTION

Parasympathetic neurons and methods of making and using the same are provided. For example, a method of making parasympathetic neurons (parasymN) can include culturing of Schwann Cell Progenitors (SCPs) in chemically-defined parasymN differentiation media. In some embodiments, the parasymN differentiation media comprises one or more of GDNF, BDNF, CNTF, and FBS. In some embodiments the media is free from NGF. A preferred parasymN differentiation media includes B27, L-Glutamine, FBS, GDNF, BDNF, CNTF, ascorbic acid, dbcAMP, and retinoic acid, optionally wherein the media is Neurobasal medium further comprising B27, L-Glutamine, 1% FBS, 25 ng/ml GDNF, 25 ng/ml BDNF, 25 ng/ml CNTF, 200 μM ascorbic acid, 0.2 mM dbcAMP and 0.125 μM retinoic acid, or a variation thereof with, e.g., 20%, 10%, 5%, etc., more or less of the foregoing ingredients. In some embodiments, the cells are cultured for about two or more weeks.

Some embodiments, include preparation of SCPs from Neural Crest cells (NCC). Preparation of SCPs can include culturing NCC in a chemically-defined SCP differentiation media. The SCPs can be cultured as spheroids. In some embodiments, SCPs are cultured in SCP differentiation media for about 6-14 days. The SCP differentiation media can include, for example, (i) B27, L-Glutamine, FGF2, dbcAMP, and NRG1, optionally wherein the media is Neurobasal medium further comprising B27, L-Glutamine, 10 ng/ml FGF2, 100 μM dbcAMP, and 20 ng/mlNRG1, or a variation thereof with, e.g., 20%, 10%, 5%, etc., more of less of the foregoing ingredients, or (ii) N2, B27, BSA, GlutaMAX, (β-mercaptoethanol, CT 99021, SB431542, and NRG1, optionally wherein the media is DMEM/F12 and Neurobasal medium (1:1 mix) further comprising 1× N2, 1× B27, 0.005% BSA, 2 mM GlutaMAX, 0.11 mM f3-mercaptoethanol, 3 μM CT 99021, and 20 μM SB431542, and wherein 50 ng/mL NRG1 is only added after 6 days, or a variation thereof with, e.g., 20%, 10%, 5%, etc., more of less of the foregoing ingredients.

In some embodiments, cells are cultured on a substrate coated with Polyornithine (PO)/laminin (LM)/fibronectin (FN).

The methods can further include preparing NCC from stem cells such as embryonic stems cells or induced pluripotent stem cells. The methods can including culturing the stem cells in a chemically-defined NCC induction media. In some embodiments, the NCC induction media includes a transforming growth factor beta (TGF(3)/Activin-Nodal signaling inhibitor and an activator of wingless (Wnt) signaling. In more particular embodiments, the NCC induction media includes BMP4, SB431542, and CHIR99021, optionally wherein the induction media is Essential 6 medium further including 0.4 ng/ml BMP4, 10 μM SB431542 and 300 nM CHIR99021 for days 0-1. Essential 6 medium further including 10 μM SB431542 and 0.75 μM CHIR99021 for days 2 on, or a variation thereof with, e.g., 20%, 10%, 5%, etc., more of less of the foregoing ingredients.

In some embodiments, the NCC are cultured in NCC induction media for 10-12 days.

Any of the cells can human cells, or derived from human tissue.

In preferred embodiments, the methods induce differentiation of about 50% of the cells into parasymN. The parasymN can be characterized by one or more of a neuron-like cell morphology, the appearance of well-developed neurite bundles, expression of one or more autonomic markers such as ASCL1, PHOX2B, PRPH, and/or CHRNA3, one or more parasympathetic markers such as ChAT, VAChT, ChT, and/or NPY2R, reduced expression of SOX/0 e.g., relative to SCPs and/or SCs, predominate expression of ChAT relative TH, e.g., compared to symNs, HOX 1-5 positive, optionally except HOX 2, one or more cholinergic muscarinic receptors (MusR), preferably at least M2 and/or M4, functionally activated by bethanechol (BeCh), functionally inactivated by atropine, insensitivity to 6-OHDA, and ability to build functional connectivity to and/or innervate target tissues.

Thus, induced cells characterized by one or more of a neuron-like cell morphology, the appearance of well-developed neurite bundles, expression of one or more autonomic markers such as ASCL1, PHOX2B, PRPH, and/or CHRNA3, one or more parasympathetic markers such as ChAT, VAChT, ChT, and/or NPY2R, reduced expression of SOX/0 e.g., relative to SCPs and/or SCs, predominate expression of ChAT relative TH, e.g., compared to symNs, HOX 1-5 positive, optionally except HOX 2, one or more cholinergic muscarinic receptors (MusR), preferably at least M2 and/or M4, functionally activated by bethanechol (BeCh), functionally inactivated by atropine, insensitivity to 6-OHDA, and ability to build functional connectivity to and/or innervate target tissues, optionally, but preferably prepared according the disclosed methods, are also provided.

In some embodiments, the induced cell population includes PRPH neurons, wherein 70%, 75%, 80%, 85%, 90%, 95%, or more of PRPH neurons express ChAT.

Methods of enriching the parasymN are also provided and can include, for example, FACS and/or immunopanning. Some of the methods include isolating parasymN from other cells in the culture. Other cells may include one or more of stem cells, NCC, SCP, and SC.

Conditioned media formed by the induced cells, and compositions including induced parasymN cells and/or conditioned media thereof are also provided. In some embodiments, the composition includes a matrix or substrate for the cells.

Therapeutic and non-therapeutic methods of treatment are also provided. Such methods can include administering the subject an effective amount of the induced cells, conditioned media harvested therefrom, or the composition thereof. In some embodiments, the subject has a disorder of neurons, optionally parasympathetic neurons, a neurodegenerative disorder, an autoimmune disease, and inflammatory disorder or condition, or a combination thereof. In some embodiments, the subject has or had Familial Dysautonomia, Sjogren's syndrome, SARS-CoV-2 infection, a condition of parasympathetic nervous system (PSNS), or an autonomic neuropathy.

Screening methods are also provided. For example, a method of screening for compounds can include contacting parasymN with one or more compounds and selecting the compound if it increases one or more functions of the cells or reduces one or more functions of the cells.

### BRIEF DESCRIPTION OF THE DRAWINGS

FIG. 1 is a schematic diagram showing the differentiation workflow to make symNs, SCs, and parasymNs from NCCs.

FIGS. 2A-2J are a schematic diagram showing Schwann cell (SC) differentiation via the neural crest (NC) cell and Schwann cell progenitor (SCP) stage (FIG. 2A), and SCP characterization for parasymN neuron induction; fold change (FC) of marker gene expression relative to DO determined by RT-qPCR from day 10-30 including PAX3 (FIG. 2B) and GAP43 (FIG. 2C) in the developing SCP, CD56 in immature Schwann cell (iSC) (FIG. 2D), and MPZ (FIG. 2E) and S100 (FIG. 2F) in SCs. n=4 biological replicates. FIGS. 2G-2I demonstrate bulk RNAseq performed to compare D10 NC and D16 SCP. Distinct cellular identity and upregulated genes involved in Schwann cell development pathways were found in D16 SCP. SCP genes were upregulated in D16 SCP compared to D10 NC (FIG. 2J).

FIGS. 3A-3D are a schematic diagram showing replating timepoints for parasymN differentiation (FIG. 3A); and table showing main ingredients of five parasymN differentiation media conditions (con) used at every replating timepoint (FIG. 3B). FIG. 3C shows single axon from neurons in each condition in FIG. 3B. FIG. 3D shows quantification and comparison of axon differentiation in FIG. 3C.

FIGS. 4A-4G are graphs showing fold change (FC) of marker gene expression relative to DO determined by RT-qPCR analysis of day 30 parasymNs for ANS markers including Asc11, PHOX2B, PRPH, and CHRNA3 (FIG. 4A) and parasymN markers including ChAT, VAChT, ChT, NPY2R, and HMX3 (FIG. 4B). n=3-5 biological replicates. Graphs showing fold change (FC) of SOX10 expression between SCs and parasymNs (FIG. 4C) and ChAT/TH ratio (FIG. 4D) between symNs and parasymNs on day 30. Student two-tailed t-test. n=3-6 biological replicates. Graphs showing percentage of cells ChAT out of PRPH neurons (ChAT/PRPH) and ChAT out of DAPI (ChAT/DAPI) (FIG. 4E); a cartoon illustration shows the alignment of HOX genes in symN system and parasymN system (FIG. 4F); and a graph showing fold change (FC) of HOX genes relative to DO determined by RT-qPCR analysis of day 30 parasymNs. n=4 biological replicates. Error bars=SEM. Scale bars represent 100 μm (FIG. 4G). **p<0.01, ****P<0.0001.

FIGS. 5A-5L are a graph showing paraymN activity (relative MFR: mean firing rate) in unstimulated (Ctrl) or nicotine (1 μM) treated SCP-derived neurons, measured by MEA (FIG. 5A). Student two-tailed t-test. n=4 biological replicates. Graph showing acetylcholine (ACh) concentration (pmole/400×10³ cells) in parasymM culture media measured by ELISA (FIG. 5B). n=3 biological replicates. Graph showing fold change (FC) of muscarinic receptors (MusRs) relative to DO determined by RT-qPCR analysis of parasymNs (FIG. 5C). Student two-tailed t-test. Data of M2/4 were pooled and compared to M1/3/5. n=3-4 biological replicates. Graph showing relative MFR in control and bethanechol (BeCh, 1 μM)-treated cells (FIG. 5D) or atropine (1 μM)-treated cells (FIG. 5E) measured by MEA. Student two-tailed t-test. n=3 biological replicates. Graph showing relative MFR of vehicle, paraymN, and symN cells over a period of 12 hours after treated with 6-OHDA (100 μM) (FIG. 5F). Values of parasymNs or symNs were normalized by their own vehicle treated groups as shown in grey. Two-way ANOVA followed by Sidak multiple comparisons. n=3-4 biological replicates. Schematic illustration of hPSC-parasymNs and hPSC-CMs co-culture (FIG. 5G). Bar graph showing relative beats/min (cardiac action potential) in control and nicotine (1 μM) treated cells of the co-culture measured by MEA (FIG. 5H). Student two-tailed t-test. n=6 biological replicates. Error bars=SEM. Scale bars represent 100 μm. *p<0.05, **p<0.01, ***p<0.001, ****P<0.0001. Bar graph showing a coculture of CM with optogenetic parasymN which can be specifically activated by blue light (FIG. 5I) compared to the nicotine treatment (FIG. 5H). FIGS. 5J-5K show quantification of western blot 12 hours after treating parasymN/symN with 6-OHDA (FIG. 5J). Cell death markers PUMA/c-Cas3 were increased only in symN (FIG. 5K). FIG. 5L is graph showing percentage of cells after immunostaining for 24 hours after treating parasymN/symN with 6-OHDA. Cell death marker c-Cas3 was increased only in symN (FIG. 5L). Microscopy revealed that SymN also showed damaged neural morphology, while parasymN was almost intact.

FIGS. 6A-6B are bar graphs showing percent ChAT+ cells differentiated from a hESC line (MEL1, male) and four hiPSC lines (healthy control 652, 11 y.o. female, Cpl, 21 y.o. female, hDFn, male, neonate and C1, 35 y.o. female) based on the immunofluorescence images (FIG. 6A). n=3 biological replicates. FIG. 6B shows fold change (FC) of gene expression relative to DO hPSC of neurons differentiated from the protocol by Kim et al. n=4 biological replicates for parasymN markers. Error bars=SEM. FIG. 6C is a bar graph showing qPCR results that support the increased ChAT expression upon BF treatment, which represented increased maturity.

FIGS. 7A-7J are graph showing fold change (FC) of gene expression relative to DO for autonomic neural markers in control (ctrl-ESC-H9 and ctrl-iPSC-C1, pooled) and FD (FD-iPSC-S2) parasymNs (FIG. 7A). Multiple unpaired Student's t-test. n=3-4 biological replicates. Graph showing mean firing rate (Hz) in control (ctrl-ESC-H9 and ctrl-iPSC-C1, pooled) and FD (FD-iPSC-S2) parasymNs from Day 20 to Day 60 of differentiation using MEA analysis (FIG. 7B). Two-way ANOVA followed by Sidak multiple comparisons. n=3-5 biological replicates. Graph showing fold changes of mean firing rate at Day 60 of FD (FD-iPSC-S2) symNs and parasymNs relative to control (ctrl-ESC-H9 and ctrl-iPSC-C1, pooled) symNs and parasymNs (FIG. 7C). Student two-tailed t-test. n=4-11 biological replicates. Cartoon illustration of the effects of acetylcholine (ACh) released by parasymNs to cardiomyocyte (CM) and smooth muscle (SMC) (FIG. 7D). Graphs showing beating rate (times/min) of hPSC-CMs (FIG. 7E) and contractivity (times/min) of hPSC-SMCs (FIG. 7F) co-cultured with control (ctrl-ESC-H9) and FD (FD-iPSC-S2) parasymNs. Student two-tailed t-test. n=4 biological replicates. FIG. 7G is a bar graph accessing ELP1 splicing which was found to be impaired in parasymN. FIGS. 7H-7J show bulk RNAseq performed to compare ctrl and FD parasymN. Distinct cellular identity and upregulated genes involved in cell stress and hyperactivity pathways were found in parasymN.

FIGS. 8A-8G are graph showing fold change (FC) of gene expression relative to DO for cholinergic signaling markers including ChAT, VAChT, and ChT in control (ctrl-ESC-H9 and ctrl-iPSC-C1, pooled) and FD (FD-iPSC-S2) parasymNs (FIG. 8A). Multiple unpaired Student's t-test. n=3-5 biological replicates. Graph showing fold change (FC) of gene expression relative to DO for signal transduction markers including CHRNA3, CHRNB4, CHRM2, and CHRM4 in control (ctrl-ESC-H9 and ctrl-iPSC-C1, pooled) and FD (FD-iPSC-S2) parasymNs (FIG. 8B). Multiple unpaired Student's t-test. n=3-5 biological replicates. Graph showing fold change (FC) of gene expression relative to DO for NPY2R in control (ctrl-ESC-H9 and ctrl-iPSC-C1, pooled) and FD (FD-iPSC-S2) parasymNs (FIG. 8C). Student two-tailed t-test. n=3-5 biological replicates. Schematic illustration of conditional medium treatments (FIG. 8D). Graphs showing relative MFR of FD (FD-iPSC-S2) symNs and parasymNs treated with conditional media from control (ctrl-ESC-H9 and ctrl-iPSC-C1, pooled) and FD (FD-iPSC-S2) parasymNs and symNs using MEA analysis (FIG. 8E & FIG. 8F). One-way ANOVA followed by Tukey's multiple comparisons. n=5-9 biological replicates. Cartoon illustration shows that in FD peripheral nervous system (PNS), both parasympathetic nervous system (PSNS) and sympathetic nervous system (SNS) are hyperactive, but SNS is stronger. It also shows that the crosstalk between PSNS and SNS is impaired (FIG. 8G). Error bars=SEM. Scale bars represent 100 μm. *p<0.05, **p<0.01, ****P<0.0001.

FIGS. 9A-9G are cartoon illustration of how SARS-CoV-2 infection leads to an imbalanced renin-angiotensin-aldosterone system (RAAS), which leads to an imbalanced autonomic nervous system (ANS) that worsens the cardiovascular complications observed in COVID-19 patients (FIG. 9A). Graph showing fold change (FC) of gene expression relative to DO for angiotensin receptors (AGTR1/2) in parasymNs (FIG. 9B), n=3 biological replicates. Graph showing fold change (FC) of MFR in control (DMSO) and angiotensin II (AngII)-treated parasymNs using MEA analysis (FIG. 9C). Student two-tailed t-test. n=10 biological replicates. Schematic illustration of the potential anti-inflammatory effect of parasymN conditional media in an anti-inflammation assay (FIG. 9D). Graph showing ROS level of hPSC-CMs with each treatment shown as CM-H2DCFDA intensity measured by ELISA (FIG. 9E). One-way ANOVA followed by Tukey's multiple comparisons. n=4 biological replicates. Graph showing cardiac activity (beating rate timnes/min) of hPSC-CMs with each treatment measured by MEA (FIG. 9F). One-way ANOVA followed by Tukey's multiple comparisons. n=3 biological replicates. Graph showing beating variability of hPSC-CMs with each treatment measured by MEA (FIG. 9G). One-way ANOVA followed by Tukey's multiple comparisons. n=3 biological replicates. Error bars=SEM. *p<0.05, **p<0.01, ***P<0.001. FC=fold change. MFR=mean firing rate.

FIGS. 10A-10G are schematic illustration of the antibody-based complement-dependent cytotoxicity assay (FIG. 10A). Graph showing positive cells per neural cluster in Control IgG, P1 IgG, and P2 IgG (FIG. 10B). Three clusters in each biological replicate were analyzed from 3-4 biological replicates. One-way ANOVA followed by Tukey's multiple comparisons. Graph showing fold change (FC) of MFR in parasymNs treated with control and SS patient IgG using MEA analysis (FIG. 10C). One-way ANOVA followed by Tukey's multiple comparisons. n=4 biological replicates. Graph showing acetylcholine in the extracellular space in parasymN cultures treated with control and SS patient IgG measured by ELISA (FIG. 10D). One-way ANOVA followed by Tukey's multiple comparisons. n=3 biological replicates. Graph showing acetylcholinesterase (AChE) activity from parasymNs treated with control and SS patient IgG measured by ELISA (FIG. 10E). One-way ANOVA followed by Tukey's multiple comparisons. n=4 biological replicates. Graph showing ROS level of parasymNs treated with control and SS patient IgG was shown as CM-H2DCFDA intensity measured by ELISA (FIG. 10F). One-way ANOVA followed by Tukey's multiple comparisons. n=3 biological replicates. Cartoon illustration summarized that in SS patients, parasymNs can be targeted by auto-antibodies (auto-Abs), which decreases parasymN activity. ACh extracellular level is increased, and AChE is reduced, possibly due to the autoimmune response (FIG. 10G). Error bars=SEM. *p<0.05, ***p<0.001, ****P<0.0001. FC=fold change. MFR=mean firing rate.

FIGS. 11A-11N are schematic illustration of parasymN and white adipocyte co-culture (FIG. 11A); and schematic illustration of white adipocyte differentiation from 3T3-L1 pre-adipocyte cells (FIG. 11B). Graph showing Oli red concentration (OD at 492 nm) measured by ELISA in Day 0 and differentiated adipocytes from 3T3-L1 cells (FIG. 11C). Student two-tailed t-test. n=3-4 biological replicates. Graph showing glycerol release (μg/ml) from differentiated white adipocytes in control and with 10 μM isoproterenol (iso) (FIG. 11D). Paired student two-tailed t-test. n=4 biological replicates. Error bars=SEM. *p<0.05, ****P<0.0001. Graph showing FABP4 intensity in the adipocytes with or without parasymNs (FIG. 11E). Student two-tailed t-test. n=6 biological replicates. Graph showing lipid droplet area in the adipocytes with or without parasymNs using LipidSpot staining (FIG. 11F). Student two-tailed t-test. n=4 biological replicates. Graph showing nucleus area (pixel) in the adipocytes with or without parasymNs (FIG. 11G). 10 nuclei in each biological replicate were analyzed from 3 biological replicates. Student two-tailed t-test. Graph showing glycerol release (μg/ml) from adipocytes representing the lipolytic activity measured by ELISA in control and nicotine-treated parasymNs (FIG. 11H). 1 μM nicotine was applied to activate parasymNs. Multiple unpaired Student's t-test. n=4-6 biological replicates. Graph showing normalized glycerol release from adipocytes with or without parasymNs in the presence or absence of 10 μM isoproterenol (iso) (FIG. 11I). One-way ANOVA followed by Tukey's multiple comparisons. n=4 biological replicates. FIG. 11J and FIG. 11K are graphs demonstrating the result of a glucose uptake assay performed to confirm the reduced glucose uptake in adipocyte after coculturing with or without parasymNs. Student two-tailed t-test. n=4. Error bars=SEM. *p<0.05, ** p<0.01, ****P<0.0001. FC=fold change. FIG. 11L is a plot showing qPCR results of adipocyte marker expressions. FIG. 11M is a plot showing qPCR analysis for another parasymN marker (VAChT) expression in the coculture. FIG. 11N is plot showing qPCR analysis for lipolysis markers (HSL & KGLL) performed to support the lipolysis assay.

FIGS. 12A-12E show comparison of symN and parasymN development. FIG. 12A is plot showing the results of migration assay for D14 symN progenitor and D16 parasymN progenitor (SCP) where SCP were more migratory than D14 cells. FIG. 12B-12D show results of bulk RNAseq performed to compare D14 symN progenitor and D16 SCP. Distinct cellular identity and upregulated genes involved in cell adhesion and neural development pathways were found in D14 cells. FIG. 12E is a PCA plot showing developmental trajectory of parasymN from D10 NC and D16 SCP.

FIGS. 13A-13B demonstrate that non-neural contaminating cell types in parasymN differentiation can be purified. FIG. 13A illustrates that cell cycle inhibitor AraC was added during the differentiation. FIG. 13B is a plot quantifying the results after immunostaining showed that contaminating cells are reduced.

FIGS. 14A-14E demonstrate dry mouth syndrome in SS patient using parasymN and mouse salivary cell coctulture. FIG. 14A is a plot showing the percentage of cells with granules that increased in coculture, indicating increased salivary cell maturation with parasymNs. FIG. 14B is a plot showing calcium imaging for salivary cell only upon nicotine stimulation. FIG. 14C is a plot showing calcium imaging for coculture upon nicotine stimulation, and salivary cell activity increased due to parasymN activation by nicotine. FIG. 14D demonstrates the results after Amylase ELISA that showed that amylase synthesis was not changed in the coculture, which fit current understanding in vivo. FIG. 14E is a plot showing calcium activity in coctulture was reduced when the culture was treated with SS patient serum. This recapitulates the dry mouth symptom in the patient.

### DETAILED DESCRIPTION OF THE INVENTION

I. Definitions

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

As used herein, the term “a population of cells” or “a cell population” refers to a group of at least two cells. In non-limiting examples, a cell population can include at least about 10, at least about 100, at least about 200, at least about 300, at least about 400, at least about 500, at least about 600, at least about 700, at least about 800, at least about 900, at least about 1000 cells. The population may be a pure population comprising one cell type, such as a population of parasympathetic neurons, or a population of undifferentiated stem cells. Alternatively, the population may comprise more than one cell type, for example a mixed cell population such as one including parasympathetic neurons and other cells, e.g., one or more undifferentiated stem cells, Schwann Cell Progenitors, Schwann Cells, and sympathetic neurons.

As used herein, the term “stem cell” refers to a cell with the ability to divide for indefinite periods in culture and to give rise to specialized cells. A stem cell refers to a stem cell that is from a human.

As used herein, the term “embryonic stem cell” refers to a primitive (undifferentiated) cell that is derived from preimplantation-stage embryo, capable of dividing without differentiating for a prolonged period in culture, and are known to develop into cells and tissues of the three primary germ layers. A human embryonic stem cell refers to an embryonic stem cell that is from a human. As used herein, the term “human embryonic stem cell” or “hESC” refers to a type of pluripotent stem cells derived from early stage human embryos, up to and including the blastocyst stage, that is capable of dividing without differentiating for a prolonged period in culture, and are known to develop into cells and tissues of the three primary germ layers.

As used herein, the term “embryonic stem cell line” refers to a population of embryonic stem cells which have been cultured under in vitro conditions that allow proliferation without differentiation for up to days, months to years.

As used herein, the term “totipotent” refers to an ability to give rise to all the cell types of the body plus all of the cell types that make up the extraembryonic tissues such as the placenta.

As used herein, the term “multipotent” refers to an ability to develop into more than one cell type of the body.

As used herein, the term “pluripotent” refers to an ability to develop into the three developmental germ layers of the organism including endoderm, mesoderm, and ectoderm.

As used herein, the term “induced pluripotent stem cell” or “iPSC” refers to a type of pluripotent stem cell, similar to an embryonic stem cell, formed by the introduction of certain embryonic genes (such as a OCT4, SOX2, and KLF4 transgenes) (see, for example, Takahashi and Yamanaka Cell 126, 663-676 (2006), herein incorporated by reference) into a somatic cell, for examples, CI 4, C72, and the like. An induced pluripotent stem cell may be prepared from any fully (e.g., mature or adult) or partially differentiated cell using methods known in the art. For example, but not by way of limitation, an induced pluripotent stem cell may be prepared from a fibroblast, such as a human fibroblast; an epithelial cell, such as a human epithelial cell; a blood cell such as a lymphocyte or hematopoietic cell or cell precursor or myeloid cell, such as a human lymphocyte, hematopoietic cell or cell precursor or human myeloid cell; or a renal epithelial cell, such as a human renal epithelial cell. In certain non-limiting embodiments, an induced pluripotent stem cell contains one or more introduced reprogramming factor associated with producing pluripotency. In certain non-limiting embodiments a human induced pluripotent stem cell is not identical to a human embryonic pluripotent stem cell.

As used herein, the term “somatic cell” refers to any cell in the body other than gametes (egg or sperm); sometimes referred to as “adult” cells.

As used herein, the term “somatic (adult) stem cell” refers to a relatively rare undifferentiated cell found in many organs and differentiated tissues with a limited capacity for both self-renewal (in the laboratory) and differentiation.

Such cells vary in their differentiation capacity, but it is usually limited to cell types in the organ of origin.

As used herein, the term “neuron” refers to a nerve cell, the principal functional units of the nervous system. A neuron consists of a cell body and its processes—an axon and one or more dendrites. Neurons transmit information to other neurons or cells by releasing neurotransmitters at synapses.

As used herein, the term “proliferation” refers to an increase in cell number.

As used herein, the term “undifferentiated” refers to a cell that has not yet developed into a specialized cell type.

As used herein, the term “differentiation” refers to a process whereby an unspecialized embryonic cell acquires the features of a specialized cell such as a heart, liver, or muscle cell. Differentiation is controlled by the interaction of a cell's genes with the physical and chemical conditions outside the cell, usually through signaling pathways involving proteins embedded in the cell surface.

As used herein, the term “directed differentiation” refers to a manipulation of stem cell culture conditions to induce differentiation into a particular (for example, desired) cell type, such as neurons or a subtype thereof such as sympathetic neurons (symNs) and parasympathetic neurons (parasymNs). As used herein, the term “directed differentiation” in reference to a stem cell typically refers to the use of small molecules, growth factor proteins, and other growth conditions to promote the transition of a stem cell from the pluripotent state into a more mature or specialized cell fate (e.g., neurons or a subtype thereof such as parasymN).

As used herein, the term “inducing differentiation” in reference to a cell refers to changing the default cell type (genotype and/or phenotype) to a non-default cell type (genotype and/or phenotype). Thus, “inducing differentiation in/of a stem cell” refers to inducing the stem cell (e.g., stem cell) to divide into progeny cells with characteristics that are different from the stem cell, such as genotype (e.g., change in gene expression as determined by genetic analysis such as a microarray) and/or phenotype (e.g., change in expression of a protein, such as one or more markers.

As used herein, the term “cell culture” refers to a growth of cells in vitro in an artificial medium for research or medical treatment.

As used herein, the term “culture medium” refers to a liquid that covers cells in a culture vessel, such as a Petri plate, a multi-well plate, and the like, and contains nutrients to nourish and support the cells. Culture medium may also include growth factors added to produce desired changes in the cells.

As used herein, the term “contacting” cells with a compound refers to placing the compound in a location that will allow it to touch the cell. The contacting may be accomplished using any suitable methods. For example, contacting can be accomplished by adding the compound to a tube of cells. Contacting may also be accomplished by adding the compound to a culture medium comprising the cells. Each of the compounds can be added to a culture medium comprising the cells as a solution (e.g., a concentrated solution).

Alternatively or additionally, the compounds as well as the cells can be present in a formulated cell culture medium.

As used herein, the term “in vitro” refers to an artificial environment and to processes or reactions that occur within an artificial environment. In vitro environments exemplified, but are not limited to, test tubes and cell cultures.

As used herein, the term “in vivo” refers to the natural environment (e.g., an animal or a cell) and to processes or reactions that occur within a natural environment, such as embryonic development, cell differentiation, neural tube formation, etc.

As used herein, the term “expressing” in relation to a gene or protein refers to making an mRNA or protein which can be observed using assays such as microarray assays, antibody staining assays, and the like.

As used herein, the term “marker” or “cell marker” refers to gene or protein that identifies a particular cell or cell type. A marker for a cell may not be limited to one marker, markers may refer to a “pattern” of markers such that a designated group of markers may identity a cell or cell type from another cell or cell type.

As used herein, the term “derived from” or “established from” or “differentiated from” when made in reference to any cell disclosed herein refers to a cell that was obtained from (e.g., isolated, purified, etc.) a parent cell in a cell line, tissue (such as a dissociated embryo, or fluids using any manipulation, such as, without limitation, single cell isolation, cultured in vitro, treatment and/or mutagenesis using for example proteins, chemicals, radiation, infection with virus, transfection with DNA sequences, such as with a morphogen, etc., selection (such as by serial culture) of any cell that is contained in cultured parent cells. A derived cell can be selected from a mixed population by virtue of response to a growth factor, cytokine, selected progression of cytokine treatments, adhesiveness, lack of adhesiveness, sorting procedure, and the like.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

As used herein, the term “an effective amount” or “effective amounts” refers to an amount of a molecule or other composition such as cell that is sufficient to achieve a desired effect. In some examples, the amount is effective in directing the in vitro differentiating of stem cells into a population of differentiated cells expressing one or more desired markers. In certain embodiments, the population of differentiated cells includes cells expressing one or more desired marker.

As used herein, “transformed” and “transfected” encompass the introduction of a nucleic acid (e.g., a vector) into a cell by a number of techniques known in the art.

### II. Methods of Making Differentiated Parasympathetic Neurons

The Examples below exemplify parasympathetic neurons (parasymNs) that exhibit typical molecular and physiological properties generated from various human embryonic stem cell (hESC) and human induced pluripotent stem cell (hiPSC) lines. Accordingly, compositions and methods for making parasympathetic neurons (parasymNs) are provided. Methods of using the cells and compositions related thereto are also provided and include, but are not limited to, study and/or treatment of Parkinson's disease, Alzheimer's disease, hypertension, multiple system atrophy, diabetic autonomic neuropathy, spinal cord injury, COVID-19, and familial dysautonomia (FD).

An exemplary method is exemplified in the experiments below, and the disclosed compositions and methods may include any one or more compositions or methodologies described therein. Specific time points for culturing, culture conditions, and concentrations and dosages of culture supplements are provided below and in the working Examples, and can be utilized separately or together in any combination or sub-combination. Also specifically disclosed are plus/minus ranges up to 20%, e.g., up to 10%, up to 5%, or up to 1% of each given value for e.g., time points for culturing, culture conditions, and concentrations and dosages of culture supplements. Likewise, in some embodiments any one or more of the reagents and time points to methods as exemplified in the disclosure or experiments below are varied up or down by any integer value within and including 15 times more or less, or any specific range there between, of the utilized concentration/amount/value.

A. Starting Cells

Typically the methods include in vitro differentiation of cells into parasympathetic neurons (parasymNs). The starting cells can be stems cells or Schwann or Schwann-like cells, such as induced Schwann cell progenitor cells. In some embodiments the starting cell type is stem cells that proceed to parasympathetic neurons (parasymNs) through an intermediate state in which the cells are Schwann or Schwann-like cells, preferably Schwann cell progenitor cells.

1. Stem Cells

In certain embodiments, the stem cells are human stem cells. Non-limiting examples of human stem cells include human embryonic stem cells (hESC), human pluripotent stem cell (hPSC), human induced pluripotent stem cells (hiPSC), human parthenogenetic stem cells, primoridal germ cell-like pluripotent stem cells, epiblast stem cells, F-class pluripotent stem cells, somatic stem cells, cancer stem cells, or any other cell capable of lineage specific differentiation. In certain embodiments, the human stem cell is a human embryonic stem cell (hESC). In certain embodiments, the human stem cell is a human induced pluripotent stem cell (hiPSC). In certain embodiments, the stem cells are non-human stem cells. Non-limiting examples of non-human stem cells include non-human primate stem cells, rodent stem cells, dog stem cells, cat stem cells, horse stem cells, pig stem cells, etc. In certain embodiments, the stem cells are pluripotent stem cells. In certain embodiments, the stem cells are embryonic stem cells. In certain embodiments, the stem cells are induced pluripotent stem cells.

The cells can be autologous, e.g. derived from the subject, or syngenic. Allogeneic cells can also be isolated from antigenically matched, genetically unrelated donors (identified through a national registry), or by using cells obtained or derived from a genetically related sibling or parent.

2. Schwann Cells

Schwann cells (SCs) are a type of glial cell that surrounds neurons, keeping them alive and sometimes covering them with a myelin sheath, and are the major glial cell type in the peripheral nervous system. They play important roles in the development, maintenance, function, and regeneration of peripheral nerves.

Neural crest cells (NCCs) are SOX10⁺ early embryonic progenitor cells that are highly migratory and give rise to a large variety of cell types, including all peripheral nervous system cells, melanocytes, and peripheral glia. It has been previously established an efficient and defined NC differentiation protocol that was shown to give rise to sensory enteric, and symNs. These NCCs can be used to derive Schwann cells (SCs), that showed identities that are similar to primary SCs and were capable of myelination (Majd, H., et al., bioRxiv 2022.08.16.504209; doi.org/10.1101/2022.08.16.504209 (2022); Cell Stem Cell, 30(5):632-647.e10, (2023); doi: 10.1016/j.stem.2023.04.006).

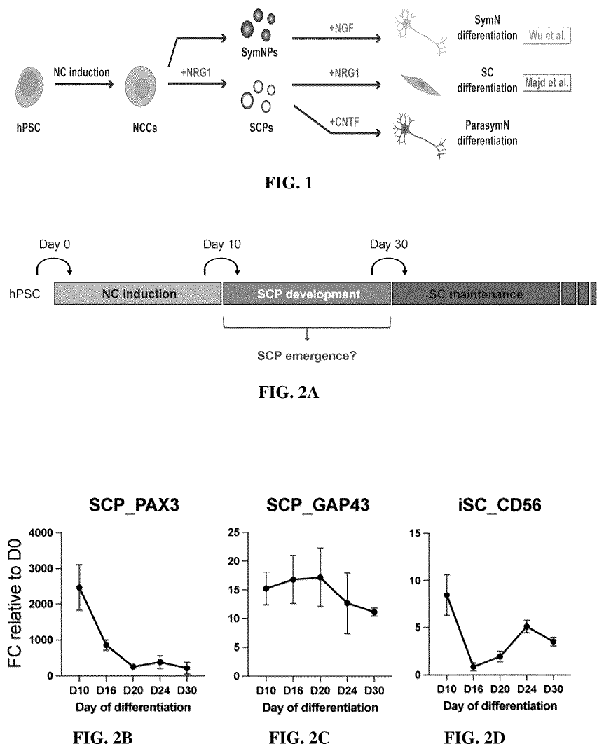

In some embodiments, the starting cell type or an intermediate cell type proceeding from a stem cell or neural crest cell starting cell type, is Schwann cell progenitor (SCP) cells. See, e.g., FIG. 1.

B. Culture Steps and Differentiation Factors

Culture steps, differentiation factors, and methods of in vitro differentiation cells into parasympathetic neurons (parasymNs) are provided. Any of the methods can include one or more of (1) inducing stem cells (e.g., ESCs or IPSCs) to differentiate into neural crest cells (NC), (2) inducing NCs to differentiate into SCPs.

1. NC Differentiation

Prior to differentiation, plates are typically coated with a substrate such as Geltrex or vitronectin or Matrigel, and used to culture stem cells, followed by induction to differentiate into neural crest cells (NC cells or NCC).

Cells are fed and cultured over time and may or may not include one, two, three, or more replatings. In some embodiments, the cells are stabilized, e.g., after thawing, e.g., by splitting 2-3× after thawing. The cells can be cultured in a monolayer, neurospheres, or a combination thereof. In some embodiments, a feeder layer is utilized, e.g., mouse fibroblasts. In some embodiments, the methods are also free from a feeder layer.

The disclosed methods and compositions for preparing NC cells can include one or more compositions or methods or steps thereof disclosed in U.S. Patent Application Nos. 2019/0093074 and 2022/0195386, U.S. Pat. No. 9,453,198, Wu, H. F., Zeltner, et al., “Efficient Differentiation of Postganglionic Sympathetic Neurons using Human Pluripotent Stem Cells under Feeder-free and Chemically Defined Culture Conditions,” J. Vis. Exp. (159), e60843, doi:10.3791/60843 (2020), each if which is specifically incorporated by reference herein in its entirety.

In some embodiments, stem cells are plated on basement membrane matrix at day 0, feed with differentiation media for about 10 days (e.g., about 8, 9, 10, 11, or 12 days) beginning on day 0 or day 1. In some embodiments, two differentiation medias are used, a first differentiation media for about 1 day, followed by a second differentiation media for the remainder of the NC differentiation period.

The methods can include transforming growth factor beta (TGF(3)/Activin-Nodal signaling inhibition and/or wingless (Wnt) signaling activation and/or inhibition of Rho-binding kinase (i.e., ROCK inhibition). The methods can include BMP supplementation, e.g., BMP4. The methods thus can include culturing or otherwise contacting the cells with one or more TGFβ/Activin-Nodal signaling inhibitors and/or one or more Wnt signaling activators and/or one or more ROCK inhibitors and/or one or more BMPs. Typically the inhibitor(s) and/or activator(s) and/or BMP(s) are used in a suitable combination and effective amounts and for sufficient duration to differentiate stems cells, and/or induce them to form and/or maintain them as NC cells, e.g., as exemplified herein.

Non-limiting examples of inhibitors of TGFβ/Activin-Nodal signaling are disclosed in WO2011/149762, and are otherwise known in the art. In certain embodiments, the inhibitor of TGFβ/Activin-Nodal signaling is a small molecule selected from the group consisting of SB431542, derivatives thereof, and mixtures thereof. “SB431542” refers to a molecule with a number CAS 301836-41-9, a molecular formula of C₂₂K₈N₄O₃, and a name of 4-[4-(1,3-benzodioxol-5-yl)-5-(2-pyridinyl)-1H-imidazol-2-yl]-benzamide. In some embodiments, the inhibitor of TGFβ/Activin-Nodal signaling, e.g., SB431542, is used in a concentration ranging from 5 μM to 10 μM inclusive. A particularly preferred concentration is about 10 μM, or 10 μM. In some embodiments when two differentiation medias are used, this molarity can be used in both a first differentiation media and a second differentiation.

The ROCK inhibitor can be any inhibitor as long as it inhibits the function of Rho-binding kinase. Examples of the ROCK inhibitor include GSK269962A (Axon medchem), Fasudil hydrochloride (Tocris Bioscience), Y-27632 and H-1152 (all from, Wako Pure Chemical). A preferred example includes Y-27632. In some embodiments, the ROCK inhibitor, e.g., Y-27632, is used in a concentration ranging from 5 μM to 10 μM inclusive. A particularly preferred concentration is about 10 μM, or 10 μM. In some embodiments when two differentiation medias are used, about 10 μM, or 10 μM, of Y-27632 molarity is used in the first differentiation media and absent in second differentiation.

Non-limiting examples of bone morphogenic proteins (BMPs) include BMP2, BMP4, BMP6, and BMP7. In some embodiments, the BMP, e.g., BMP4, is used in a concentration ranging from 0 ng/ml to 5 ng/ml inclusive. A particularly preferred concentration is about 1 ng/ml, or 1 ng/ml. BMP, e.g., BMP4 can be important, and is preferably used at the lower end of a range that gives the desired results, e.g., as discussed herein such as efficiently obtaining NC. Thus, in some embodiments the BMP, e.g., BMP4 is titrated, particularly down, to determine the best concentration, which may vary somewhat between different cell types, e.g., different hPSC lines. In some embodiments, when two differentiation medias are used, BMP is present in the first differentiation media and absent from the second differentiation media.

In certain embodiments, the activator of Wnt signaling lowers GSK3P for activation of Wnt signaling. Thus, the activator of Wnt signaling can be a GSK3β inhibitor. Non-limiting examples of activators of Wnt signaling or GSK3β inhibitors are disclosed in WO2011/149762, and Calder et al., J Neurosci. 2015 Aug. 19; 35(33):11462-81, which are incorporated by reference in their entireties, and are otherwise known in the art. In certain embodiments, the activator of Wnt signaling is a small molecule selected from the group consisting of CHIR99021, derivatives thereof, and mixtures thereof “CHIR99021” (also known as “aminopyrimidine” or “343-(2-Carboxyethyl)-4-methylpyrrol-2-methylidenyl]-2-indolinone”) refers to IUPAC name 64244-(2,4-dichlorophenyl)-5-(4-methyl-1H-imidazol-2-yl)pyrimidin-2-yla-mino) ethylamino)nicotinonitrile. In some embodiments, the activator of Wnt signaling, e.g., CHIR99021, is used in a concentration ranging from 100 nM to 500 nM from days 0-2 and/or 0.25 μM to 1.5 μM from day 2 on, inclusive. In embodiments, when two differentiation medias are used, the activator of Wnt signaling, e.g., CHIR99021, is used at a concentration of 300 nM in the first differentiation media, and/or 0.75 μM CHIR99021 in the second differentiation media.

For example, in the Examples below, on day 0, hPSCs were replated on Geltrex (Invitrogen, A1413202)-coated plates at 125×10³ cells/cm². Day 0-1 a first differentiation medium composed of Essential 6 medium supplemented with 0.4 ng/ml BMP4, 10 μM SB431542 and 300 nM CHIR99021. From day 2 on, cells were fed with a second differentiation medium composed of Essential 6 medium supplemented with 10 μM SB431542 and 0.75 μM CHIR99021.

In another example, NCC differentiation is initiated (DO) by aspirating the maintenance medium (E8) and replacing it with a first neural crest induction medium [BMP4 (1 ng ml-1), SB431542 (10 μM), and CHIR 99021 (600 nM) in Essential 6 medium]. Subsequently, on D2 a second neural crest induction medium [SB431542 (10 μM) and CHIR 99021 (1.5 μM) in Essential 6 medium] was fed to the cultures until D12.

NC stage (e.g., days 10-12) cells can be frozen and stored for later use. In some embodiments, the CryoPause method (Wong, et al., Stem Cell Reports 9, 355-365 (2017)) is utilized.

2. Schwann Cell Progenitors

In some embodiments the methods include inducing a starting cell type to form Schwann Cell Progenitor (SCP) cells.

For example, beginning with NCC, including but not limited to those formed according to the methods described above, Schwann cells can be induced according methods that are known in the art including, but not limited to, Majd, et al., “Deriving Schwann Cells from hPSCs Enables Disease Modeling and Drug Discovery for Diabetic Peripheral Neuropathy,” bioRxiv 2022.08.16.504209; doi.org/10.1101/2022.08.16.504209, and Kim, et al., “Schwann Cell Precursors from Human Pluripotent Stem Cells as a Potential Therapeutic Target for Myelin Repair”, Stem Cell Reports 8, 1714-1726 (2017), doi: 10.1016/j.stemcr.2017.04.011, each of which is specifically incorporated by reference herein in its entirety.

SCs are thought to arise from SOX10⁺NC cells in a stepwise process. For example, based on studies in the mouse and chick embryos, NC first gives rise to SC precursors that are competent to associate with neuronal fiber bundles in the developing nerves. The associated neurons produce NRG1 which promotes the survival and further differentiation of SC precursors (SCPs) by activating ERBB3 receptors. By E13.5 of mouse development, SC precursors give rise to immature SCs which express lineage-specific markers such as GFAP, 5100 and POU3F1 while maintaining the expression of SOX10. Terminal differentiation of SCs into myelinating and non-myelinating fates continues for extended time periods and concludes only after birth.

In some embodiments, NCC, including but not limited to day 10-12 NCC prepared according to the methods discussed above, are replated as spheroids until about day 24 during which the cells are induced to form Schwann Cell Progenitors. For example, in some embodiments, NC monolayers are detached, e.g., using accutase. Cells are collected and replated as 3D spheroids, and cultured in medium supplemented with L-Glutamine, N2 and B27, an activator of Wnt signaling, e.g., CHIR99021, FGF2, and NRG1. In a particular embodiment, the media is Neurobasal media containing with L-Glutamine, FGF2 (10 ng ml-1), CHIR 99021 (3 μM), N2 supplement (10 μl ml-1), B27 supplement (20 μl m1-1), and NRG1 (10 ng/ml). Preferably, the spheroids are cultured on ultra-low-attachment plates to form free-floating 3D developing precursors. Such media can be referred to SC precursor media (SCP).

In other embodiments, colonized hPSCs can be re-plated onto growth factor-reduced Matrigel-coated culture dishes. The next day, the culture medium was switched to culture medium containing N2, B27, BSA, GlutaMAX, f3-mercaptoethanol, CT 99021, and SB431542. In a specific embodiment, the media is advanced DMEM/F12 and Neurobasal medium (1:1 mix) supplemented with 1×N2, 1×B27, 0.005% BSA, 2 mM GlutaMAX, 0.11 mM β-mercaptoethanol, 3 μM CT 99021, and 20 μM SB431542. After about 6 days of differentiation, the medium is further supplemented with NRG1 (e.g., 50 ng/mL). In some embodiments, media is changed daily. These Schwann Cell Progenitors can be routinely dissociated with Accutase treatment upon reaching 80% confluence and were expanded by additional cultivation in the foregoing differentiation media. The hPSC-derived SCPs can be generated after approximately 18 days of differentiation. The foregoing media can be used for the induction and maintenance of hPSC-derived SCPs.

As a non-limiting example, in the Examples below, day 10 NCCs were replated as spheroids in SCP medium until day 24. Spheroids were plated on PO/LM/FN coated plates without dissociation in SC differentiation medium that contains Neurobasal medium, B27, L-Glutamine, 10 ng/ml FGF2, 100 μM dbcAMP, and 20 ng/mlNRG1. SC identity was evaluated on day 30.

3. Parasympathetic Neurons (parasymNs)

Compositions and method for inducing parasymNs are provided. The parasymNs are typically derived from SCPs. Optionally, but preferably, the SCPs are prepared from NCC. Optionally, but preferably, the NCC are prepared from stem cells.

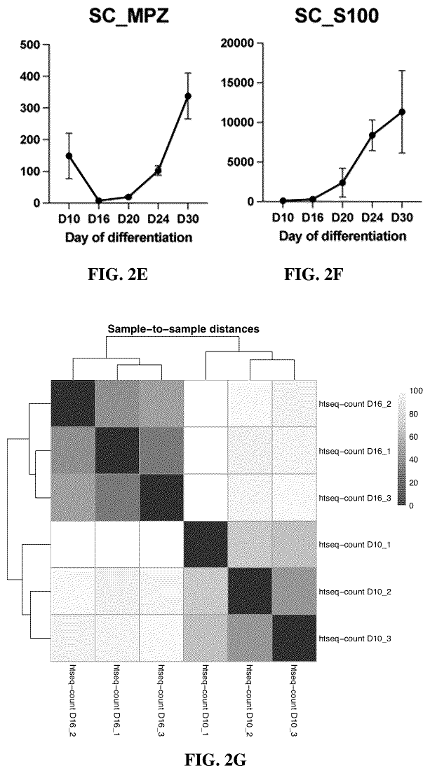

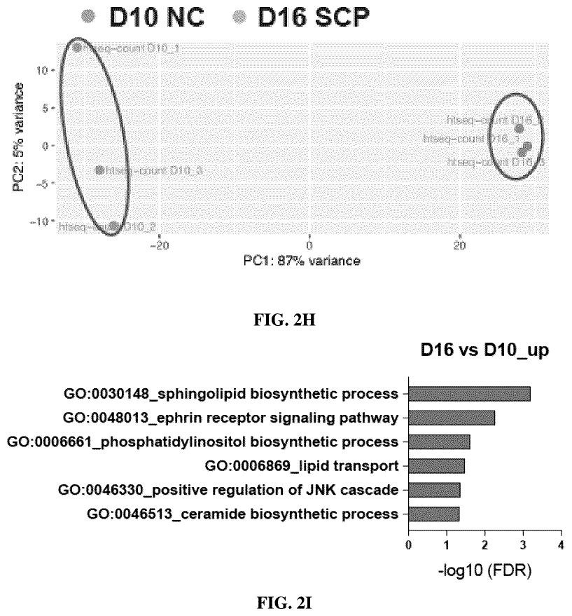

In preferred embodiments, the SCPs are multipotent, but not yet determined to the SC fate. Preferably, the cells have relatively high levels of SCP markers (such as PAX3/GAP43)) and relatively lower levels of immature SC makers (e.g., iSC, CD56), and differentiated SC markers (e.g., MPZ, S100β), e.g., compared to SCs. Results presented below show that SCP genes decreased from day 16-20, accompanied by increasing iSC marker, which dropped after day 24, while SC genes were still increasing (FIGS. 2B-2F). These results demonstrate a clear progress of SC differentiation and indicate that the proper timing to start parasymN induction can be between day 6-14 after plating spheroids (e.g., 16-24 days after initiation of NCC induction). Further experiments indicated that replating before day 6 (about day 6 from NCC induction, when SCP induction is initiated at day 10) induced neuron-like cell morphology, and the appearance of well-developed neurite bundles. Thus, in some embodiments, ParasymNs induction is initiated in SCPs about 4-14 days after plating NCC spheroids (i.e., 14-26 days after inducing NCC differentiation from stem cells).

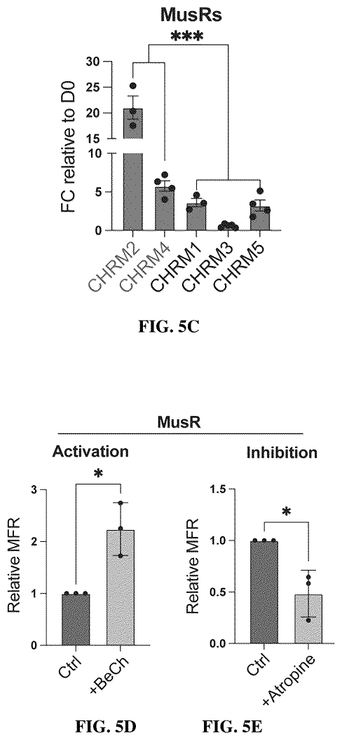

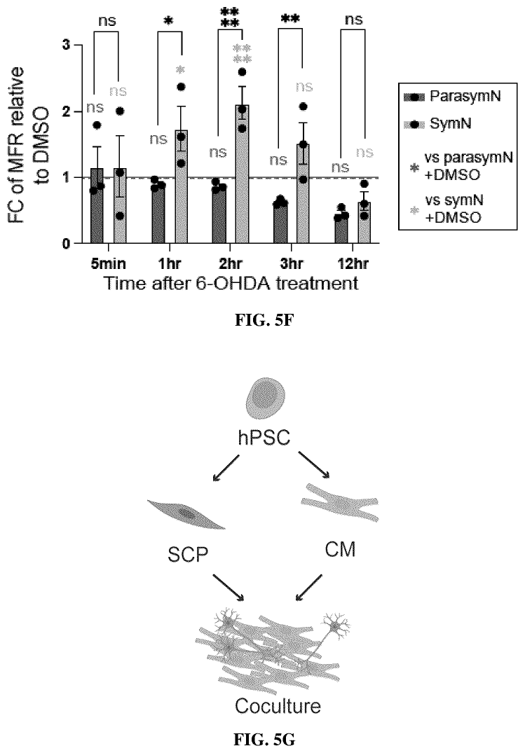

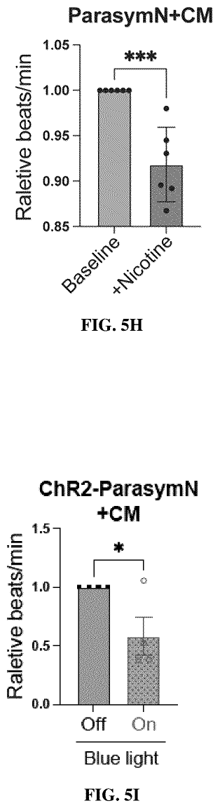

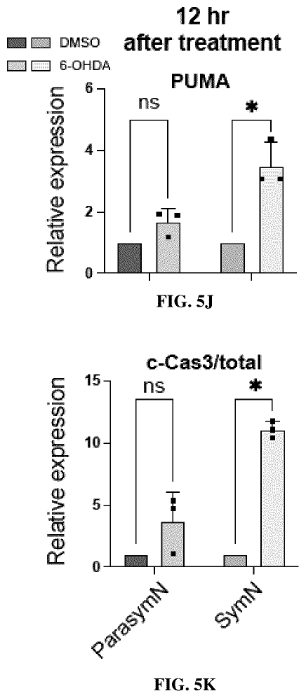

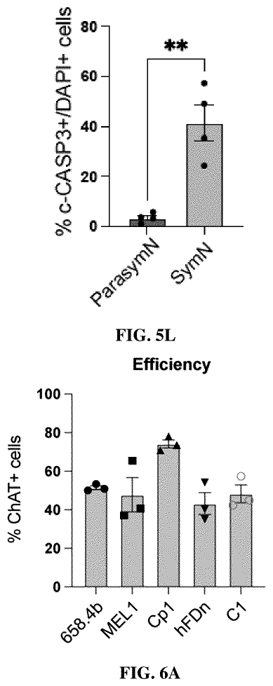

ParasymNs are typically induced using a combination of one or more of Glial Cell Derived Neurotrophic Factor (GDNF), Brain-derived Neurotrophic Factor (BDNF), Ciliary Neurotrophic Factor (CNTF), Fetal Bovine Serum (FBS), and Nerve Growth Factor (NGF). Preferably the media includes all of GDNF, BDNF, CNTF, and FBS. See, e.g., FIG. 5B which provided non-limiting combinations. NGF is believed to be important for symN survival, and results below show parasymN can be induced without it. Thus, in some embodiments, NGF is excluded from the media. This may reduce potential symN contamination from NCCs that are not fully differentiated. In some embodiments, the neural supplement BrainFast is added. For example, SCP spheroids can be plated on e.g., PO/LM/FN-coated plates and cultured in media containing B27, L-Glutamine, FBS, GDNF, BDNF, CNTF, ascorbic acid, dbcAMP, and retinoic acid. In a more particular embodiments, the media is Neurobasal medium with B27, L-Glutamine, 1% FBS, 25 ng/ml GDNF, 25 ng/ml BDNF, 25 ng/ml CNTF, 200 μM ascorbic acid, 0.2 mM dbcAMP, and 0.125 μM retinoic acid optionally, but preferably, added freshly every feeding.

ParasymNs can be differentiated over about two weeks and can be maintained in parasymN differentiation medium by halfway feeding weekly.

In preferred embodiments, parasymNs are characterized by one or more of a neuron-like cell morphology, the appearance of well-developed neurite bundles, expression of one or more autonomic markers such as ASCL1, PHOX2B, PRPH, and/or CHRNA3, one or more parasympathetic markers such as ChAT, VAChT, ChT, and/or NPY2R, reduced expression of SOX/0 e.g., relative to SCPs and/or SCs, predominate expression of ChAT relative TH, e.g., compared to symNs, HOX 1-5 positive, optionally except HOX 2 (e.g., HOX 1-5⁺, but HOX²⁻, also referred to HOX 1⁺, 3⁺, 4⁺,5⁺, HOX²⁻), one or more cholinergic muscarinic receptors (MusR), preferably at least M2 and/or M4, functionally activated by bethanechol (BeCh), functionally inactivated by atropine, insensitivity to 6-OHDA, ability to build functional connectivity to and/or innervate target tissues e.g., cardiomyocytes (CMs) (e.g., by forming neurocardiac junctions, optionally nodal structures that are sensitive to nicotine), adipocytes, etc. and/or other biochemical, morphological, and/or functional features discussed in more detail in the experiments below. Markers can be measured quantitatively or qualitatively in any suitable way, for example, gene expression can be assessed by RT-PCR, protein expression can be assessed by Western Blot, immunofluorescence, etc.

In some embodiments, the methods are carried out under conditions that yield about 50% or more ChAT⁺ parasymNs optionally but preferably in combination with very high differentiation specificity as indicated by almost all (e.g., at least 70%, 75%, 80%, 85%, 90%, 95%, or more) PRPH⁺ neurons expressing ChAT.

In a specific embodiment, the method follows the protocol as used in the Examples below. ParasymNs are differentiated from SCPs. Day 10 NCCs were dissociated using accutase and aggregated to make spheroids on ultra-low attachment plates in SCP medium containing Neurobasal medium, B27, L-Glutamine, 3 μM CHIR99021, 10 ng/ml FGF2, and 10 ng/ml NRG1. On day 16, SCP spheroids were dissociated using accutase and replated on PO/LM/FN-coated plates at 100×10³ cells/cm² in parasymN differentiation medium is Neurobasal medium including B27, L-Glutamine, 1% FBS, 25 ng/ml GDNF, 25 ng/ml BDNF, 25 ng/ml CNTF, 200 μM ascorbic acid, 0.2 mM dbcAMP and 0.12511M retinoic acid (add freshly every feeding).

Typically the method yields functional parasymNs. Function can be measured using any suitable method, including those discussed in the experiments below.

C. Selection and/or Isolation

In some embodiments, the method includes steps to selection for and/or isolation of parasymNs. Such steps can include, but are not limited to, FACS or immunopanning.

Immunopanning (Sloan, et al., Neuron 95, 779-790.e6 (2017), specifically incorporated by reference herein in its entirety) is a gentle antibody-based purification technique that can be used to segregate the different parasymNs from other cells, e.g., non-parasymNs including, but not limited to, SCP, SCs, and symNs. This method allows the binding of specific cells from a mix to a dish pre-coated with antibodies against cell surface proteins. The cells of interest attach to the antibody and the following wash and dissociation steps are much gentler compared to FACS. Suitable cells markers that can be targeted by antibodies for immunopanning parasymNs are discussed elsewhere herein and are otherwise known in the art. Preferably the cell marker is an extracellular marker.

### III. Cell Populations and Compositions Thereof

A. Cell Populations Cells prepared according the disclosed methods are also provided. Thus, the presently disclosed subject matter provides compositions including a population of differentiated parasymNs produced by the in vitro differentiation methods described herein. In certain non-limiting embodiments, the differentiated cells are prepared from embryonic pluripotent stem cells, such as human embryonic pluripotent stem cells. In certain non-limiting embodiments, the differentiated cells are prepared from induced pluripotent stem cells, such as induced human pluripotent stem cells.

Compositions including a population of in vitro differentiated cells are also provided. In some embodiments, at least about 50%, (e.g., at least about 55%, at least about 60%, at least about 65%, at least about 70%, at least about 75%, at least about 80%, at least about 85%, at least about 90%, at least about 95%, at least about 99%, or at least about 99.5%) of the population of cells express one or more markers of parasymNs, and/or lack one or more markers of pluripotent cells and/or an undesired differentiated cell population (e.g., SCPs, SCs, symNs, etc.), which can be measured a gene and/or protein expression.

Markers of pluripotent stem cells, partially differentiated and differentiated parasymNs neurons, SCs, SCPs, and symNs that can be used to distinguish cells types are discussed herein, such as in the Examples below, and are known in the art.

See, e.g., U.S. Published Application Nos. 2022/0195386 and 2019/0093074 each of which is specifically incorporated by reference herein in its entirety.

Non-limiting examples of stem cell markers include OCT4, NANOG, SOX2, LIN28, SSEA4 and SSEA3.

Non-limiting markers of SCP and/or SCs include SOX10, PAX3/GAP43, iSC, CD56, MPZ, and S1000.

Non-limiting markers for symNs include TH.

In some embodiments, less than about 50% (e.g., less than about 45%, less than about 40%, less than about 35%, less than about 30%, less than about 25%, less than about 20%, less than about 15%, less than about 10%, less than about 5%, less than about 4%, less than about 3%, less than about 2%, less than about 1%, less than about 0.5%, or less than about 0.1%) of the population of cells express one or more markers of pluripotent cells and/or an undesired partially or fully differentiated cell population, e.g., SCP, SC, symNs, etc.

Non-limiting markers of parasymNs include ASCL1, PHOX2B, PRPH, CHRNA3, ChAT, VAChT, ChT, NPY2R, HOX1, HOX3, HOX4, HOX5, MusR M2 and MusR M4.

In some embodiments, more than about 40% (e.g., more than about 45%, more than about 50%, more than about 55%, more than about 60%, more than about 65%, more than about 70%, more than about 75%, more than about 80%, more than about 85%, more than about 90%, or more than about 95%) of the population of cells express one or more markers of parasymNs cells.

B. Genetically Modified Cells

In some embodiments, the disclosed cells do not contain any gene or genetic modification. Additionally or alternatively, the cells can also be free from transfection with nucleic acid constructs. For example, as exemplified below, the differentiation programs described herein can be carried out without gene modification or transgene expression.

However, in some embodiments, the precursor stem cells and or the differentiated cells have been genetically modified and/or include one or more nucleic acid expression constructs. Gene modifications typically refer to modification of the cell's genome can include induced by any suitable means, e.g., triplex-forming molecules, pseudocomplementary oligonucleotides, CRISPR/Cas, zinc finger nucleases, TALENs, viral mediated integration, etc. These technologies are known in the art can be used to make modifications to the cells ranging from point mutations to deletions and insertions of e.g., expression constructs. Additional or alternative, the cells may optionally be transfected with transient or permanently nucleic acid expression constructs in the form of e.g., mRNA, viral vectors, plasmids, and other extrachromosomal means of gene expression. Such genetic modifications and expression constructs can be used for a variety of purposes including, but not limited to, facilitating or enhancing preparation of the precursor (e.g., stem) cells, preparation of the differentiated cells (e.g., sensor neurons), and/or for gene therapy.

In particularly preferred embodiments containing a genetic modification or extrachromosomal expression construct, the cells are enhanced for use in gene therapy applications. Gene therapy is a technique that modifies a person's genes to treat or cure disease. Gene therapies can work by several mechanisms, for example, (1) replacing a disease-causing gene with a healthy copy of the gene; (2) inactivating a disease-causing gene that is not functioning properly; or (3) introducing a new or modified gene into the body to help treat a disease. For example, in some embodiments, the cells are used to a treat a disease, such a genetic disorder, that includes reduced expression of a wildtype protein and/or expression of mutant protein. Such cells can be modified to, for example, reverse a detrimental genetic mutation and/or express or overexpress a compensatory protein.

Thus, in some embodiments, cells prepared according to the disclosed methods are infected, transfected or otherwise modified to express an expression construct. For example, constructs can be inserted into vectors for expression in cells. As used herein, a “vector” is a replicon, such as a plasmid, phage, or cosmid, etc., into which another DNA segment may be inserted so as to bring about the replication of the inserted segment. Vectors can be expression vectors. An “expression vector” is a vector that includes one or more expression control sequences, and an “expression control sequence” is a DNA sequence that controls and regulates the transcription and/or translation of another DNA sequence. Nucleic acids in vectors can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence.

Suitable expression vectors include, without limitation, plasmids and viral vectors derived from, for example, herpes viruses, cytomegalo virus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen Life Technologies (Carlsbad, CA).

C. Conditioned Media

Results in the experiments below show that the disclosed parasymNs can secrete molecules into the culture, that can have therapeutic effects. For example, hPSC-parasymN-conditioned media ameliorated the ROS accumulation and improved cardiomyocyte functionality under the inflammatory conditions. Thus, media conditioned by the disclosed parasymNs cells, further compositions formed therefrom, and methods of use thereof are also provided.

Conditioned media can include one or more of the collection of proteins that contain a signal peptide and are processed via the endoplasmic reticulum and Golgi apparatus through the classical secretion pathway, proteins shed from the cell surface, intracellular proteins released through non-classical secretion pathway, extracellular vesicles including but not limited to exosomes. These secreted materials include numerous enzymes, growth factors, cytokines and hormones or other soluble mediators. Conditioned media may influence, e.g., cell growth, differentiation, invasion and angiogenesis by regulating cell-to-cell and cell-to-extracellular matrix interactions of parasymNs and/or other cell types. In some embodiments, conditioned media from parasymNs can be used to rescue a deficiency in a subject in need thereof, e.g., caused by a loss or dysfunction of parasympathetic neurons, particularly where cell therapy is not necessary, or is impractical, or impossible.

The conditioned media can be prepared by culturing a sufficient number of parasymNs for a sufficient period of time to secrete the desire factors at the desired amount to achieve the therapeutically goals. In some embodiments, the culturing is carried out for 1, 2, 3, 4, 5, 6, 7, 8, 9, 10, or more days. The conditioned media can be used without modification (e.g., neat), or the factors therein can be concentrated and/or sub-fractioned.

D. Compositions

Compositions including cell populations made according to the disclosed methods and/or conditioned media made therefrom are also provided. In certain non-limiting embodiments, the composition includes a biocompatible scaffold or matrix, for example, a biocompatible three-dimensional scaffold that facilitates tissue regeneration when the cells are implanted or grafted to a subject. In certain non-limiting embodiments, the biocompatible scaffold comprises extracellular matrix material, synthetic polymers, cytokines, collagen, polypeptides or proteins, polysaccharides including fibronectin, laminin, keratin, fibrin, fibrinogen, hyaluronic acid, heparin sulfate, chondroitin sulfate, agarose or gelatin, and/or hydrogel. (See, e.g., U.S. Publication Nos. 2015/0159135, 2011/0296542, 2009/0123433, and 2008/0268019, the contents of each of which are incorporated by reference in their entireties).

In certain embodiments, the composition is a pharmaceutical composition that includes a pharmaceutically acceptable carrier, excipient, diluent or a combination thereof. In certain embodiments, the compositions can be used to treat or prevent a neuron disorder or a disease characterized by neuron loss or dysfunction. In certain embodiments, the compositions can be used for preventing and/or treating a disorder of parasympathetic neurons, a neurodegenerative disease or disorder, an autoimmune disease or disorder, or an infectious disease, particularly those that impact neuronal health and/or function such as SARS-CoV-2.

The compositions can be provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof. Sterile injectable solutions can be prepared by incorporating the compositions of the presently disclosed subject matter, in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the presently disclosed cells.

Viscosity of the compositions, if desired, can be maintained at the selected level using a pharmaceutically acceptable thickening agent. Methylcellulose can be used because it is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Compositions should be selected to be chemically inert and will not affect the viability or efficacy of the presently disclosed cells and/or active agents in the conditioned media. This will present no problem to those skilled in chemical and pharmaceutical principles, or problems can be readily avoided by reference to standard texts or by simple experiments, from this disclosure and the documents cited herein.

### IV. Methods of Use

Methods of using the disclosed differentiated cells and compositions thereof are also provided. The methods include investigation of disease etiology, treatment of diseased subjects, and testing of compounds for e.g., therapeutic or toxic effect.

A. Methods of Treatment

In certain embodiments the in vitro differentiated cells and/or a composition thereof can be used for preventing and/or treating a disorder of neurons, particularly parasympathetic neurons. Such methods of preventing and/or treating a disorder of neurons and/or a neurodegenerative disorder can include administering to a subject in need thereof a therapeutically effective amount of the presently disclosed differentiated cells, conditioned media, or a composition form therefrom.

The cells or compositions thereof can be administered or provided systemically or locally to a subject for preventing and/or treating the disease or disorder. In certain embodiments, cells, conditioned media, or a composition formed therefrom are directly injected into an organ of interest (e.g., an organ affected by a disorder of neurons and/or a neurodegenerative disorder).

The cells and compositions thereof can be administered in any physiologically acceptable vehicle. Pharmaceutical compositions cell or a composition thereof and a pharmaceutically acceptable carrier are also provided. In some embodiments, the cells or compositions are administered via localized orthotropic (OT) injection, local application, systemic injection, intravenous injection, or parenteral administration.

The cells, media, or a composition thereof can be administered to a subject in a therapeutically effective amount. A “therapeutically effective amount” is an amount sufficient to affect a beneficial or desired clinical result upon treatment. A therapeutically effective amount can be administered to a subject in one or more doses. In terms of treatment, a therapeutically effective amount is an amount that is sufficient to palliate, ameliorate, stabilize, reverse or slow the progression of the disorder of neurons and/or neurodegenerative disorder, or otherwise reduce the pathological consequences of the disorder of neurons and/or neurodegenerative disorder. The therapeutically effective amount is generally determined by the physician on a case-by-case basis and is within the skill of one in the art. Several factors are typically taken into account when determining an appropriate dosage to achieve a therapeutically effective amount. These factors include age, sex and weight of the subject, the condition being treated, the severity of the condition and the form and effective concentration of the cells administered.

The quantity of cells or amount of conditioned media to be administered will vary for the subject being treated and the condition being treated. In certain embodiments, from about 1×10⁴ to about 1×10¹⁰, from about 1×10⁴ to about 1×10⁵, from about 1×10⁵ to about 1×10⁹, from about 1×10⁵ to about 1×10⁶, from about 1×10⁵ to about 1×10⁷, from about 1×10⁶ to about 1×10⁷, from about 1×10⁶ to about 1×10⁸, from about 1×10⁷ to about 1×10⁸, from about 1×10⁸ to about 1×10⁹, from about 1×10⁸ to about 1×10¹⁰, or from about 1×10⁹ to about 1×10¹⁰ the presently disclosed cells are administered to a subject. The precise determination of what would be considered a therapeutically effective dose may be based on factors individual to each subject, including their size, age, sex, weight, and condition of the particular subject. Dosages can be readily ascertained by those skilled in the art from this disclosure and the knowledge in the art.

In some embodiments, the cells are transiently or genetically modified as discussed above. Such cells can serve as a combination of both cell and gene therapy. The appropriate gene modification and/or recombinant expression construct can be selected by the practitioner based on the disease or disorder to be treated. For example, in some embodiments the cells are modified to enhance treatment of a monogenic disorder. Monogenic disorders (monogenic traits) are caused by variation in a single gene and are typically recognized by their striking familial inheritance patterns. In such embodiments, the genetic modification may reverse or correct or otherwise compensate for the mutated gene by, for example, expressing a wildtype copy of the mutant gene.

A non-limiting example is Familial Dysautonomia (FD). FD is a monogentic disorder that is caused by a homozygous point mutation in the ELP1 gene. This leads to missplicing of the elongator complex protein 1 (ELP1) protein. The mutant protein is degraded, and patients thus have dramatically reduced levels of ELP1 protein. This is particularly prevalent in sensory and sympathetic tissues. Thus, in some embodiments, the cells are genetical modified to express one or more wildtype copies of ELP1 protein, e.g., by having one or more endogenous gene and/or heterologous expression constructs that encode and express wildtype ELP1 protein (e.g., an active copy of the ELP1 gene). Accordingly, for example, ELP1-gene delivery into hPSC-derived parasymN and transplanted into the DRG of patients may be used as a therapy for FD.

In other embodiments, the compositions are used to treat an autoimmune disorder, particularly where parasympathetic neurons are attached, e.g., Sjorgen's syndrome.

In other embodiments, the compositions are used to treat an infectious disease, e.g., that cause neural damage or dysfunction, particularly to parasympathetic neurons, including, but not limited to SARS-CoV-2.

In other embodiments, the compositions are used a therapeutic or non-therapeutic methods of enhancing development and/or maturation of tissue that is innervated by parasympathetic neurons, e.g., cardiovascular tissue, adipose tissue, etc. In some embodiments, the compositions are used to regulate white adipose tissue (WAT) maturation and functionality, for example, by negatively regulating WAT lipolysis and/or decreasing glucose uptake in adipocytes.

B. Drug Screening

The disclosed cells and compositions thereof are useful to investigate the activity or applicability of one or more test compounds to treat or alleviate one or more symptoms of a neuronal or neurodegenerative disease or disorder.

In a typical embodiment, cells are cultured under conditions suitable to induce differentiation of the desired cell population as disclosed herein. In some embodiments, the cells are isolated from a diseased subject, or healthy cells are treated with a disease-inducing compound, to form a diseased, dysfunctional, or a defective cell model. One or more test compounds can be applied to cultured differentiated cells and evaluated for the ability to treat one or more symptoms of the diseased, dysfunctional, or defective cells. The symptom or symptoms can be specific to the disease state being studied, or can be of a generally nature.

In some embodiments, healthy cells are utilized, and compounds are tested for toxicity and/or the ability to further improve one or more wildtype functions.

Physiological, phenotypic, morphological, or molecular symptoms and other markers of the cells can be monitored over time.

C. Diseases and Disorders

Diseases and disorders that can be the subject of the disclosed methods include those that effect parasympathetic neurons and may be neurodevelopmental and/or neurodegenerative. In some embodiments the disease or disorder is a condition of parasympathetic nervous system (PSNS), an autonomic neuropathy, neurodegenerative disease, autoimmune disease, infectious disease, developmental disease or disorder, or injury.

Exemplary PSNS and autonomic diseases and disorders include, but are not limited to, type 2 diabetes, congenital and genetic conditions including but not limited to amyloidosis and Familial Dysautonomia, loss of control of bladder and/or bowels, multiple system atrophy, sexual dysfunction (e.g., erectile dysfunction), orthostatic hypotension, postprandial hypotension, pure autonomic failure, afferent baroreflex failure, and trauma.

Exemplary neurodegenerative diseases include, but are not limited to, amyotrophic lateral sclerosis (ALS), Huntington's disease (HD), and muscular dystrophies, problems with the way the nervous system develops, such as spina bifida, degenerative diseases, where nerve cells are damaged or die, such as Parkinson's disease (PD) and PD-related disorders, meningitis, prion diseases such as Creutzfeldt-Jakob Disease, corticobasal degeneration, frontotemporal dementia, cognitive impairment including mild cognitive impairment and HIV-related cognitive impairment, motor neuron diseases (MND), spinocerebellar ataxia (SCA), spinal muscular atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, monomelic amyotrophy, multiple system atrophy, multiple system atrophy with orthostatic hypotension (Shy-Drager Syndrome), Multiple Sclerosis (MS), neurodegeneration (e.g., with brain iron accumulation), opsoclonus myoclonus, posterior cortical atrophy, primary progressive aphasia, progressive supranuclear palsy, vascular dementia, progressive multifocal leukoencephalopathy, dementia with Lewy Bodies, lacunar syndromes, hydrocephalus, Wernicke-Korsakoff's syndrome, post-encephalitic dementia, cancer and chemotherapy-associated cognitive impairment and dementia, and depression-induced dementia, Guillain-Barre syndrome, and pseudodementia.

In some embodiments, the disease or disorder is not primarily or exclusively a central nervous system (CNS) disease or disorder.

The results below show that parasympathetic neurons may be a direct target of autoimmune diseases, particularly where autoantibodies are directed against parasymN marker such as a MusR. The results also show that parasymN have an anti-inflammatory activity, particularly against TNF-α and IL-6 released by immune cells. Thus, in some embodiments, an autoimmune disease is treated, particularly one in which parasympathetic neurons are directed targeted by the disease such as Sjogren's syndrome. In other embodiments, an inflammatory disorder or condition is treated, particularly one characterized by increased TNF-α and IL-6, such following infection, e.g., with SARS-CoV-2.

Representative inflammatory or autoimmune diseases and disorders that may be treated include, but are not limited to, rheumatoid arthritis, systemic lupus erythematosus, alopecia areata, anklosing spondylitis, antiphospholipid syndrome, autoimmune Addison's disease, autoimmune hemolytic anemia, autoimmune hepatitis, autoimmune inner ear disease, autoimmune lymphoproliferative syndrome (alps), autoimmune thrombocytopenic purpura (ATP), Behcet's disease, bullous pemphigoid, cardiomyopathy, celiac sprue-dermatitis, chronic fatigue syndrome immune deficiency, syndrome (CFIDS), chronic inflammatory demyelinating polyneuropathy, cicatricial pemphigoid, cold agglutinin disease, Crest syndrome, Crohn's disease, Dego's disease, dermatomyositis, dermatomyositis—juvenile, discoid lupus, essential mixed cryoglobulinemia, fibromyalgia—fibromyositis, grave's disease, guillain-barye, hashimoto's thyroiditis, idiopathic pulmonary fibrosis, idiopathic thrombocytopenia purpura (ITP), Iga nephropathy, insulin dependent diabetes (Type I), juvenile arthritis, Meniere's disease, mixed connective tissue disease, multiple sclerosis, myasthenia gravis, pemphigus vulgaris, pernicious anemia, polyarteritis nodosa, polychondritis, polyglancular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, sarcoidosis, scleroderma, Sjogren's syndrome, stiff-man syndrome, Takayasu arteritis, temporal arteritis/giant cell arteritis, ulcerative colitis, uveitis, vasculitis, vitiligo, Wegener's granulomatosis, and infectious diseases, including, but not limited to bacterial and viral infections such as SAR-CoV-2, and the diseases associated therewith (e.g., COVID).

In some embodiments, the compositions and methods are used to improve heath and developed, e.g., of the nervous system, particularly the parasympathetic nervous system. Such methods may be therapeutic or none therapeutic.

The disclosed invention can be further understood by the following numbered paragraphs:
1. A method of making parasympathetic neurons (parasymN) including culturing of Schwann Cell Progenitors (SCPs) in chemically-defined parasymN differentiation media.
2. The method of paragraph 1, wherein the chemically-defined parasymN differentiation media includes one or more of GDNF, BDNF, CNTF, and FBS, optionally all of GDNF, BDNF, CNTF, and FBS, and optionally free from NGF.
3. The method of paragraphs 1 or 2, wherein chemically-defined parasymN differentiation media includes B27, L-Glutamine, FBS, GDNF, BDNF, CNTF, ascorbic acid, dbcAMP, and retinoic acid, optionally wherein the media is Neurobasal medium further including B27, L-Glutamine, 1% FBS, 25 ng/ml GDNF, 25 ng/ml BDNF, 25 ng/ml CNTF, 200 μM ascorbic acid, 0.2 mM dbcAMP and 0.125 μM retinoic acid, or a variation thereof with 20%, 10%, or 5%, more or less of the foregoing ingredients.
4. The method of any one of paragraphs 1-3 including culturing the cells for about two or more weeks.
5. The method of any one of paragraphs 1-4, wherein the SCPs are prepared by culturing Neural Crest cells (NCC) in a chemically-defined SCP differentiation media.
6. The method of paragraph 5, wherein the SCPs are cultured as spheroids.
7. The method of paragraphs 5 or 6, wherein the SCPs are cultured in SCP differentiation media for about 6-14 days.
8. The method of any one of paragraphs 5-7, wherein the SCP differentiation media includes
(i) B27, L-Glutamine, FGF2, dbcAMP, and NRG1, optionally wherein the media is Neurobasal medium further including B27, L-Glutamine, 10 ng/ml FGF2, 100 μM dbcAMP, and 20 ng/ml NRG1, or a variation thereof with 20%, 10%, or 5%, more or less of the foregoing ingredients; or
(ii) N2, B27, BSA, GlutaMAX, (β-mercaptoethanol, CT 99021, SB431542, and NRG1, optionally wherein the media is DMEM/F12 and Neurobasal medium (1:1 mix) further including 1× N2, 1× B27, 0.005% BSA, 2 mM GlutaMAX, 0.11 mM (β-mercaptoethanol, 3 μM CT 99021, and 20 μM SB431542, and wherein 50 ng/mL NRG1 is only added after 6 days, or a variation thereof with 20%, 10%, or 5%, more or less of the foregoing ingredients.
9. The method of any one of paragraphs 1-8 where the cells are cultured on a substrate coated with Polyornithine (PO)/laminin (LM)/fibronectin (FN).
10. The method of any one of paragraphs 1-9, wherein the NCC are prepared by culturing stem cells, optionally embryonic stems cells or induced pluripotent stem cells, in a chemically-defined NCC induction media.
11. The method of paragraph 10, wherein the NCC induction media includes a transforming growth factor beta (TGF(3)/Activin-Nodal signaling inhibitor and an activator wingless (Wnt) signaling.
12. The method of paragraphs 10 or 11, wherein the NCC induction media includes BMP4, SB431542, and CHIR99021, optionally wherein the induction media is Essential 6 medium further including 0.4 ng/ml BMP4, 10 μM SB431542 and 300 nM CHIR99021 for days 0-1, and Essential 6 medium further including 10 μM SB431542 and 0.75 μM CHIR99021 for days 2 on, or a variation thereof with 20%, 10%, or 5%, more or less of the foregoing ingredients.
13. The method of any one of paragraphs 1-10, wherein the NCC are cultured in NCC induction media for 10-12 days.
14. The method of any one of paragraphs 1-13, wherein the method induces differentiation of about 50% of the cells into parasymN.
15. The method of any one of paragraphs 1-14, wherein the parasymN are characterized by one or more of a neuron-like cell morphology, the appearance of well-developed neurite bundles, expression of one or more autonomic markers such as ASCL1, PHOX2B, PRPH, and/or CHRNA3, one or more parasympathetic markers such as ChAT, VAChT, ChT, and/or NPY2R, reduced expression of SOX/0 e.g., relative to SCPs and/or SCs, predominate expression of ChAT relative TH, e.g., compared to symNs, HOX 1-5 positive, optionally except HOX 2, one or more cholinergic muscarinic receptors (MusR), preferably at least M2 and/or M4, functionally activated by bethanechol (BeCh), functionally inactivated by atropine, insensitivity to 6-OHDA, and ability to build functional connectivity to and/or innervate target tissues.
16. The method of any one of paragraphs 1-15, wherein the induced parasymN include PRPH⁺ neurons, wherein 70%, 75%, 80%, 85%, 90%, 95%, or more of PRPH⁺ neurons express ChAT.
17. The method of any one of paragraphs 1-16, further including enriching the parasymN.
18. The method of paragraph 17 including FACS and/or immunopanning.
19. The method of any one of paragraphs 1-18 including isolating parasymN from other cells in the culture.
20. The method of paragraph 19, wherein the other cells include one or more of stem cells, NCC, SCP, and SC.
21. A population of cells formed according to the method of any one of paragraphs 1-20.
22. Conditioned media formed by the population of cells according to paragraph 21.
23. A composition including the population of cells according to paragraph 21 and/or conditioned media of paragraph 22.
24. The composition of paragraph 23 including a matrix or substrate for the cells.
25. The composition of paragraphs 23 or 24 in a pharmaceutical composition including a pharmaceutically acceptable carrier.
26. A method of treating a subject in need thereof, including administering the subject an effective amount of the cells of paragraph 21, conditioned media of paragraph 22, or the composition of any one of paragraphs 23-25.
27. The method of paragraph 26, wherein the subject has a disorder of neurons, optionally parasympathetic neurons, a neurodegenerative disorder, an autoimmune disease, and inflammatory disorder or condition, or a combination thereof.
28. The method of paragraphs 26 or 27, wherein the subject has or had Familial Dysautonomia, Sjogren's syndrome, SARS-CoV-2 infection, a condition of parasympathetic nervous system (PSNS), or an autonomic neuropathy.
29. A method of screening for compounds including contacting cells of paragraph 21 with one or more compounds and selecting the compound if it increases one or more functions of the cells or reduces one or more functions of the cells.
30. Induced parasymN cells including one or more of a neuron-like cell morphology, the appearance of well-developed neurite bundles, expression of one or more autonomic markers such as ASCL1, PHOX2B, PRPH, and/or CHRNA3, one or more parasympathetic markers such as ChAT, VAChT, ChT, and/or NPY2R, reduced expression of SOX/0 e.g., relative to SCPs and/or SCs, predominate expression of ChAT relative TH, e.g., compared to symNs, HOX 1-5 positive, optionally except HOX 2, one or more cholinergic muscarinic receptors (MusR), preferably at least M2 and/or M4, functionally activated by bethanechol (BeCh), functionally inactivated by atropine, insensitivity to 6-OHDA, and ability to build functional connectivity to and/or innervate target tissues.
31. A method of inducing sensory neurons according to the description, experiments, figures, or other disclosure herein, or any combination thereof.
32. Cells formed according to the method of paragraph 31.
33. Conditioned media formed by the cells of paragraphs 30 or 32.
34. A composition including the cells of paragraphs 30 or 32, or the conditioned media of paragraph 33, optionally wherein the composition includes a matrix or substrate for the cells and/or is a pharmaceutical composition including a pharmaceutically acceptable carrier.
35. A method of treating a subject in need thereof, optionally wherein the subject has a disorder of neurons, optionally parasympathetic neurons, a neurodegenerative disorder, an autoimmune disease, and inflammatory disorder or condition, or a combination thereof, including administering the subject an effective amount of the cells of paragraphs 30 or 32 or conditioned media of paragraph 33, or composition of paragraph 34.
36. A method of screening for compounds including contacting cells of paragraphs 30 or 32 or composition of paragraph 34 with one or more compounds and selecting the compound if it increases one or more functions of the cells or reduces one or more functions of the cells.

### EXAMPLES

### Example 1: Methods for Deriving Parasympathetic Neurons from Human Pluripotent Stem Cells

### Materials and Methods

hPSC and iPSC Maintenance

Stem cell lines used here are listed in Zeltner, 2016 and Wu, 2022. hESC lines: WA09 and H9 Phox2B::GFP (female, NIH 0062), MEL1 (male, NIH 0139). hiPSC lines: healthy 652, 11 y.o. female, healthy Cpl, 21 y.o. female, hDFn (neonatal foreskin fibroblast-derived iPSCs, Gibco #C0045C), healthy C1, 35 y.o. female, Familila Dysautonomia FD-S2. Other cell lines: 3T3-L1 (ATCC, #CL173). Detailed stem cell maintenance was previously described (Wu, H. F. & Zeltner, N. J Vis Exp (2020)). Cells were maintained in Essential 8 medium (Gibco, A15170-01) on vitronectin coated (Thermo Fisher/Life Technologies, A14700, 5 μg/ml) cell culture plates, and passaged using EDTA (Sigma, ED2SS).

In Vitro Differentiations

NC differentiation. The detailed protocol is described in Wu, 2020(Wu, H. F. & Zeltner, N. J Vis Exp (2020)). On day 0, hPSCs were replated on Geltrex (Invitrogen, A1413202)-coated plates at 125×10³ cells/cm2. Day 0-1 medium contains Essential 6 medium (Gibco, A15165-01), 0.4 ng/ml BMP4 (PeproTech, 314-BP), 10 μM SB431542 (R&D Systems, 1614) and 300 nM CHIR99021 (R&D Systems, 4423). From day 2 on, cells were fed with medium containing Essential 6 medium, 10 μM SB431542 and 0.75 μM CHIR99021. Note that BMP4 activity varies from batch to batch. It is highly recommended to perform BMP4 titrations for each batch/lot to get the best differentiation efficiency.

ParasymN differentiation. To differentiated parasymNs from SCPs, day 10 NCCs were dissociated using accutase (Coring, AT104500) and aggregated to make spheroids on ultra-low attachment plates (Corning, 07 200 601 and 07 200 602) in SCP medium containing Neurobasal medium (Gibco, 21103-049), B27 (Gibco, 17502-048), L-Glutamine (Thermo Fisher/Gibco, 25030-081), 3 μM CHIR99021, 10 ng/ml FGF2 (R&D Systems, 233-FB/CF), and 10 ng/ml NRG1 (PeproTech, 100-03). On day 16, SCP spheroids were dissociated using accutase and replated on PO (Sigma, P3655)/LM (R&D Systems, 3400-010-01)/FN (VWR/Corning, 47743-654)-coated plates at 100×10³ cells/cm² in parasymN differentiation medium that contains Neurobasal medium, B27, L-Glutamine, 1% FBS (Atlanta Biologicals, S11150), 25 ng/ml GDNF (PeproTech, 450), 25 ng/ml BDNF (R&D Systems, 248-BD), 25 ng/ml CNTF (R&D Systems, 257-NT), 200 μM ascorbic acid (Sigma, A8960), 0.2 mM dbcAMP (Sigma, D0627) and 0.125 μM retinoic acid (Sigma, R2625, add freshly every feeding). ParasymNs are differentiated in two weeks and can be maintained in parasymN differentiation medium by halfway feeding weekly.

SymN differentiation. A detailed protocol is described in Wu, 2020, supra. Day 10 NCCs were dissociated using accutase and aggregated to generate spheroids on ultra-low attachment plates in medium containing Neurobasal medium, B27, L-Glutamine, 3 μM CHIR99021 and 10 ng/ml FGF2. On day 14, spheroids were dissociated using accutase and replated on PO/LM/FN coated plates at 100×10³ cells/cm² in symN differentiation medium that contains Neurobasal medium, B27, L-Glutamine, 25 ng/ml GDNF, 25 ng/ml BDNF, 25 ng/ml NGF, 200 μM ascorbic acid, 0.2 mM dbcAMP and 0.125 μM retinoic acid (add RA freshly every feeding).

SC differentiation. The protocol is adopted from Majd, bioRxiv 2022.08.16.504209; doi.org/10.1101/2022.08.16.504209 (2022). Briefly, day 10 NCCs were replated as spheroids in SCP medium until day 24. Spheroids were plated on PO/LM/FN coated plates without dissociation in SC differentiation medium that contains Neurobasal medium, B27, L-Glutamine, 10 ng/ml FGF2, 100 μM dbcAMP, and 20 ng/mlNRG1. SC identity was evaluated on day 30.

CM differentiation. The protocol was adopted from a previous study. Briefly, 80-90% confluent H9 ESCs were replated at a 1 to 5 ratio on Matrigel (Corning, 1:20)-coated plates. When cells reached 90% confluency, medium was changed to CDBM base medium, that contains DMEM/F12 (Gibco, 11320033), 64 mg/L ascorbic acid, 13.6 μg/L sodium selenium (Sigma, S5261), 10 μg/ml transferrin (Sigma, T3309) and Chemically Defined Lipid Concentrate (Gibco, 11905031). 5 μM CHIR99021 was added to CDBM medium on day 0. 0.6 U/ml heparin (STEMCELL Technologies, 07980) was added to CDBM medium on day 1, 5, 6. 0.6 U/ml heparin and 3 μM XAV were added to CDBM medium on day 2, 3, 4. After day 7, medium was changed to CM maintenance medium that contains CDBM base, 20 μg/ml insulin (Sigma, 1-034), and 2% FBS. CMs were replated on day 10 using accutase at 200×10³ cells/cm² on Matrigel-coated plates. For parasymN co-culture, day 16 SCPs were replated on day 15 CMs in medium that was mixed with parasymN differentiation medium and CM maintenance medium at 1:1 ratio.

SMC differentiation. The protocol is adopted from previous studies (Fattahi, F., et al. Nature 531, 105-109 (2016); Patsch, C., et al. Nat Cell Biol 17, 994-1003 (2015)). Briefly, H9 ESCs were replated at 45×10³ cells/cm² on Matrigel-coated plates in Essential 8 medium. On day 1-4, cells were fed with medium that contains Neurobasal medium, N2, B27, DMEM/F12, 8 μM CHIR, and 25 ng/ml BMP4. On day 4-6, cells were fed with medium that contains Neurobasal medium, N2, B27, DMEM/F12, 10 ng/ml PDGF-BB (Shenandoah Biotechnology, 100-18), and 2 ng/ml Activin (Peprotech, 120-14). Cells were replated on day 6 at 35×10³ cells/cm² on Matrigel-coated plates in medium that contains Neurobasal medium, N2, B27, DMEM/F12, 2 μg/ml heparin, and 2 ng/ml Activin. For parasymN co-culture, day 16 SCPs were replated on day 12 SMCs in parasymN differentiation medium.

RT-qPCR

At least 0.5×10⁶ cells were collected using Trizol (Invitrogen, 15596026) for each sample. Reverse transcription was performed from 1 μg total RNA using ISCRIPT™ Reverse Transcription Supermix (Bio-Rad, 170884). SYBR green (Bio-Rad) RT-qPCR was ran using CFX96 Touch Deep Well Real-Time PCR Detection System (Bio-Rad), and analyzed by CFX Maestro. For primers used in this study, see Table 1.

TABLE 1
Primers used in this study

| | | Catalog/ | | | |
|---|---|---|---|---|---|
| Primers | Target | Reference | Method | Sequence | |
| AGTR1 | Human | Origene | SYBR | Forward | CAGCGTCAGTTTCAACCT |
| | | (NM_004835) | | | GTACG (SEQ ID NO: 1) |
| | | | | Reverse | GCAGGTGACTTTGGCTAC |
| | | | | | AAGC (SEQ ID NO: 2) |
| AGTR2 | Human | Origene | SYBR | Forward | CCATGTTCTGACCTTCCT |
| | | (NM_000686) | | | GGATG(SEQ ID NO: 3) |
| | | | | Reverse | CGGATTAACGCAGCTGTT |
| | | | | | GGTG (SEQ ID NO: 4) |
| Ascl1 | Human | Origene | SYBR | Forward | TCTCATCCTACTCGTCGG |
| | | (NM_004316) | | | ACGA (SEQ ID NO: 5) |
| | | | | Reverse | CTGCTTCCAAAGTCCATT |
| | | | | | CGCAC (SEQ ID NO: 6) |
| CD56 | Human | Origene | SYBR | Forward | CATCACCTGGAGGACTTC |
| | | (NM_181351) | | | TACC (SEQ ID NO: 7) |
| | | | | Reverse | CAGTGTACTGGATGCTCT |
| | | | | | TCAGG (SEQ ID NO: 8) |
| ChAT | Human | Home-made | SYBR | Forward | GCCTTCTACAGGCTCCAT |
| | | | | | CG (SEQ ID NO: 9) |
| | | | | Reverse | GCTCTCACAAAAGCCAG |
| | | | | | TGC (SEQ ID NO: 10) |
| CHRM1 | Human | Origene | SYBR | Forward | CTGGCTGGTTTCCTTTGT |
| | | (NM_000738) | | | GCTC (SEQ ID NO: 11) |
| | | | | Reverse | GGAGAGGAACTGGATGT |
| | | | | | AGCAC (SEQ ID NO: 12) |
| CHRM2 | Human | Origene | SYBR | Forward | TGCTGTCACCTTTGGTAC |
| | | (NM_000739) | | | GGCT (SEQ ID NO: 13) |
| | | | | Reverse | TGGTTGGCAACAGGCTC |
| | | | | | CTTCT (SEQ ID NO: 14) |
| CHRM3 | Human | Origene | SYBR | Forward | ACGAGAGCCATCTACTC |
| | | (NM_000740) | | | CATCG (SEQ ID NO: 15) |
| | | | | Reverse | TGTCGGCTTTCCTCTCCA |
| | | | | | AGTC (SEQ ID NO: 16) |
| CHRM4 | Human | Origene | SYBR | Forward | CTCCCATGAACCTCTACA |
| | | (NM_000741) | | | CCGTG (SEQ ID NO: 17) |
| | | | | Reverse | GACGCAGAAGTAGCGGT |
| | | | | | CAAAG (SEQ ID NO: 18) |
| CHRM5 | Human | Origene | SYBR | Forward | CCACCATCACTTTTGGCA |
| | | (NM_012125) | | | CTGC (SEQ ID NO: 19) |
| | | | | Reverse | AGGTCCTTGGTTCGCTTC |
| | | | | | TCTG (SEQ ID NO: 20) |
| CHRNA3 | Human | Origene | SYBR | Forward | TGGAGACCAACCTGTGG |
| | | (NM_000743) | | | CTCAA(SEQ ID NO: 21) |
| | | | | Reverse | CAGCACAATGTCTGGCTT |
| | | | | | CCAG (SEQ ID NO: 22) |
| CHRNB4 | Human | Origene | SYBR | Forward | ATCTGGTTGCCTGACATC |
| | | (NM_000750) | | | GTGC (SEQ ID NO: 23) |
| | | | | Reverse | TTGCAGGCGCTCTTGTAG |
| | | | | | ATGG (SEQ ID NO: 24) |
| ChT | Human | Origene | SYBR | Forward | AAAAGCCGTGGCTGGGA |
| | | (NM_021815) | | | ACTGT (SEQ ID NO: 25) |
| | | | | Reverse | GCACTTGAGCATAGGTG |
| | | | | | GCTGA (SEQ ID NO: 26) |
| DBH | Human | Origene | SYBR | Forward | GCCTTCATCCTCACTGGC |
| | | (NM_000787) | | | TACT (SEQ ID NO: 27) |
| | | | | Reverse | CAGCACTGTGACCACCTT |
| | | | | | TCTC (SEQ ID NO: 28) |
| GAPDH | Human | Origene | SYBR | Forward | GTCTCCTCTGACTTCAAC |
| | | (NM_002046) | | | AGCG (SEQ ID NO: 29) |
| | | | | Reverse | ACCACCCTGTTGCTGTAG |
| | | | | | CCAA (SEQ ID NO: 30) |
| GAP43 | Human | Origene | SYBR | Forward | GAGCAGCCAAGCTGAAG |
| | | (NM_002045) | | | AGAAC (SEQ ID NO: 31) |
| | | | | Reverse | GCCATTTCTTAGAGTTCA |
| | | | | | GGCATG (SEQ ID NO: 32) |
| HOXA1 | Human | QuantiTech | SYBR | Forward | — |
| | | (QT00011963) | | | |
| | | | | Reverse | — |
| HOXB2 | Human | Home-made | SYBR | Forward | CAATCCGCCACGTCTCCT |
| | | | | | TC (SEQ ID NO: 33) |
| | | | | Reverse | CCAGGCCATCTGCAGGC |
| | | | | | (SEQ ID NO: 34) |
| HOXB3 | Human | QuantiTech | SYBR | Forward | — |
| | | (QT00214907) | | | |
| | | | | Reverse | — |
| HOXB4 | Human | QuantiTech | SYBR | Forward | — |
| | | (QT00236390) | | | |
| | | | | Reverse | — |
| HOXB5 | Human | Home-made | SYBR | Forward | CGAAATAGACGAGGCCA |
| | | | | | GCG (SEQ ID NO: 35) |
| | | | | Reverse | CGGCCCGGTCATATCATG |
| | | | | | G (SEQ ID NO: 36) |
| HOXB7 | Human | Home-made | SYBR | Forward | CGCCCTTTGAGCAGAAC |
| | | | | | CTC (SEQ ID NO: 37) |
| | | | | Reverse | CGTTTGCGGTCAGTTCCT |
| | | | | | GAG (SEQ ID NO: 38) |
| HOXC9 | Human | Frith et al. | SYBR | Forward | GCAGCAAGCACAAAGAG |
| | | (2018) | | | GA (SEQ ID NO: 39) |
| | | | | Reverse | CGTCTGGTACTTGGTGTA |
| | | | | | GGG (SEQ ID NO: 40) |
| HOXA10 | Human | Home-made | SYBR | Forward | GCGAGCCCTCGATTCGC |
| | | | | | (SEQ ID NO: 41) |
| | | | | Reverse | GAATTGCCCAGGGAATC |
| | | | | | CTTCTC (SEQ ID NO: 42) |
| HOXD11 | Human | Home-made | SYBR | Forward | CCACGGTCAACTCGGGA |
| | | | | | CCT (SEQ ID NO: 43) |
| | | | | Reverse | TTCCTACAGACCCCGCCG |
| | | | | | TG (SEQ ID NO: 44) |
| HOXD12 | Human | QuantiTech | SYBR | Forward | — |
| | | (QT00222187) | | | |
| | | | | Reverse | — |
| HOXD13 | Human | QuantiTech | SYBR | Forward | — |
| | | (QT00209062) | | | |
| | | | | Reverse | — |
| MAP2 | Human | Origene | SYBR | Forward | AGGCTGTAGCAGTCCTG |
| | | (NM_002374) | | | AAAGG (SEQ ID NO: 45) |
| | | | | Reverse | CTTCCTCCACTGTGACAG |
| | | | | | TCTG (SEQ ID NO: 46) |
| MPZ | Human | Origene | SYBR | Forward | CTATCCTGGCTGTGCTGC |
| | | (NM_000530) | | | TCTT (SEQ ID NO: 47) |
| | | | | Reverse | ACTCACTGGACCAGAAG |
| | | | | | GAGCA (SEQ ID NO: 48) |
| NPY2R | Human | Origene | SYBR | Forward | ACTCCTAGAGGTGAACT |
| | | (NM_000910) | | | GGTCC (SEQ ID NO: 49) |
| | | | | Reverse | CATGGATCACCAAGGAG |
| | | | | | TTGCC (SEQ ID NO: 50) |
| PAX3 | Human | Origene | SYBR | Forward | GGCTTTCAACCATCTCAT |
| | | (NM_181459) | | | TCCCG (SEQ ID NO: 51) |
| | | | | Reverse | GTTGAGGTCTGTGAACG |
| | | | | | GTGCT (SEQ ID NO: 52) |
| PHOX2B | Human | Frith et al. | SYBR | Forward | CTACCCCGACATCTACAC |
| | | (2018) | | | TCG (SEQ ID NO: 53) |
| | | | | Reverse | CTCCTGCTTGCGAAACTT |
| | | | | | G (SEQ ID NO: 54) |
| PRPH | Human | Home-made | SYBR | Forward | GTGCCCGTCCATTCTTTT |
| | | | | | GC (SEQ ID NO: 55) |
| | | | | Reverse | GTCACCACCTCCCCATTC |
| | | | | | CG (SEQ ID NO: 56) |
| SOX10 | Human | Home-made | SYBR | Forward | CCAGGCCCACTACAAGA |
| | | | | | GC (SEQ ID NO: 57) |
| | | | | Reverse | CTCTGGCCTGAGGGGTG |
| | | | | | C (SEQ ID NO: 58) |
| S100 | Human | Origene | SYBR | Forward | GAAGAAATCCGAACTGA |
| | | (NM_006272) | | | AGGAGC (SEQ ID NO: 59) |
| | | | | Reverse | TCCTGGAAGTCACATTCG |
| | | | | | CCGT (SEQ ID NO: 60) |
| Synaptophysin | Human | Origene | SYBR | Forward | TCGGCTTTGTGAAGGTGC |
| | | (NM_003179) | | | TGCA (SEQ ID NO: 61) |
| | | | | Reverse | TCACTCTCGGTCTTGTTG |
| | | | | | GCAC (SEQ ID NO: 62) |
| TH | Human | Origene | SYBR | Forward | GCTGGACAAGTGTCATC |
| | | (NM_199292) | | | ACCTG (SEQ ID NO: 63) |
| | | | | Reverse | CCTGTACTGGAAGGCGA |
| | | | | | TCTCA (SEQ ID NO: 64) |
| VAChT | Human | Origene | SYBR | Forward | GCTGTTTGCTTCCAAGGC |
| | | (NM_003055) | | | TATCC (SEQ ID NO: 65) |
| | | | | Reverse | GAAGGCGAACAGGACTG |
| | | | | | TAGAG (SEQ ID NO: 66) |

Western Blot

Day 30 parasymNs and symNs on 6-well plates were prepared for sample collection. One well for each sample. Cells were washed by PBS and scraped after incubating with lysis buffer contains RIPA buffer (Sigma-Aldrich, R0278, 1×), PMSF protease inhibitor (Thermo Fisher Scientific, 36978, 1 mM) and PhosSTOP phosphatase inhibitor (Sigma-Aldrich, 4906845001). Protein concentration was measured using Bradford reagent (Bio-Rad, 5000006). Proteins were loaded as 20 μg/well and ran in 12% acrylamide gel. After the transfer, nitrocellulose membranes were blocked by 5% skim milk in TBST.

Membranes were incubated with primary antibodies overnight at 4° C. The next day, membranes were washed by PBST and incubated with secondary antibodies for one hour at room temperature. Images were taken using the iBright western blot imager (Invitrogen, FL1500). Table 2 lists primary and secondary antibodies used in this study.

TABLE 2
Antibodies used in this study

| Antibodies | Brand | Catalog | Host | Dilution |
|---|---|---|---|---|
| α-actinin | Sigma | A7811 | Mouse IgG1 | 1:1000 |
| αSMA | Sigma | A5228 | mlgG2a | 1:1000 |
| AGTR1 | Proteintech | 25343-1-AP | Rabbit | 1:200 |
| AGTR2 | Novus | NBP1-77368 | Rabbit | 1:200 |
| | Biologicals | | | |
| Anti-hm lgG | Proteintech | SA00003-12 | Goat | 1:100 |
| Calponin | Abclonal | A3734 | Rabbit | 1:200 |
| ChAT | Millipore | AB144P | Goat | IF: 1:100, |
| | | | | WB: 1:1000 |
| DAPI | Sigma | D9542 | — | 1:1000 |
| FABP4 | Abcam | ab92501 | Rabbit | 1:50 |
| GAPDH | Cell Signaling | 97166S | Mouse | WB: 1:1000 |
| GFP | Abcam | ab13970 | Chicken | 1:1000 |
| NPY2R | Lifespan | LS-C120758 | Rabbit | 1:200 |
| | Biosciences | | | |
| PHOX2B | Abcam | ab183741 | Rabbit | WB: 1:1000 |
| PRPH | Santa Cruz | SC-377093/ | Mouse | IF: 1:500, |
| | Biotechnology | H0112 | IgG2a | WB: 1:1000 |
| SYP | Santa Cruz | sc-17750 | Mouse | 1:200 |
| | Biotechnology | | | |
| TH | Pel-Freez | P40101-150 | Rabbit | IF: 1:500, |
| | | | | WB: 1:1000 |
| TUJ1 | Biolegend | 802001 | Rabbit | 1:1500 |
| | Biolegend | 801201 | Mouse | 1:1500 |
| | | | IgG2a | |
| VAChT | Abclonal | A16068 | Rabbit | 1:200 |

Immunohistochemistry

Cells were fixed by 4% paraformaldehyde for 20 minutes and washed twice by PBS. Fixed cells were permeabilized by 0.3% Triton, 1% BSA and 3% goat or donkey serum in 1× PBS for 20 minutes, and incubated with primary antibodies overnight at 4° C. The next day, cells were washed and incubated with secondary antibodies for one hour at room temperature. Lastly, cells were washed and incubated with DAPI (Sigma, D9542, 1:1000) for 15 minutes at room temperature. Fluorescent images were taken using the Lionheart FX Automated Microscope.

MEA Assay

For neural activity measurements, day 16 SCPs were plated on PO/LM/FN coated MEA plates (Axion BioSystems, BioCircuit or CytoView) at 100×10³ cells/cm². For hPSC-SMs or hPSC-SMCs co-cultured with parasymNs, day 10 CMs or day 6 SMCs were plated on Matrigel-coated MEA plates at 200×10³ cells/cm2 or 35×10³ cells/cm², respectively. Activity of differentiated parasymNs or CMs/SMCs on desired timepoints was measured using a MEA plate reader (Axion BioSystems, Maestro Pro) under the neural detection mode or cardiac detection mode, respectively, according to manufacturer's instruction.

ROS Measurement

Cells were washed twice by PBS, and incubated with 10 mM CM-DCFDA (Invitrogen, C6827) in PBS for 45 minutes at 37° C. After the incubation, cells were washed twice by PBS, and the fluorescent signal was measured using a fluorometer (BioTek) at 492 nm excitation and 520 nm emission.

Sjogren's syndrome patient serum

Serum samples were purchased from Central BioHub GmbH. Considering that SS occurs mostly in women, only female samples were used for this study. One patient with peripheral nervous system involvement (patient ID: 138237, ICD-10 code: M35.06) and another with high anti-SSA level (patient ID: 153001, ICD-10 code: M35.0) were selected.

Antibody-Based Complement-Dependent Cytotoxicity Assay

Total IgG was purified using the NAb™ Spin Kits (Thermo Scientific, 89978) according to manufacturer's instruction. Day 30 parasymNs were treated with 2% healthy complement-active human serum (Innovative Research, ICSER) and 200 nM SS IgG for 72 hours. Healthy human IgG (Sigma, 14506) was used as control.

AChE Activity

After the antibody-based complement-dependent cytotoxicity assay, cell culture medium was collected for the assay or stored at −80° C. for long-term storage. AChE level was measured using the AMPLITE™ Colorimetric Acetylcholinesterase Assay Kit (AAT Bioquest, 11400) according to manufacturer's instruction.

ACh ELISA

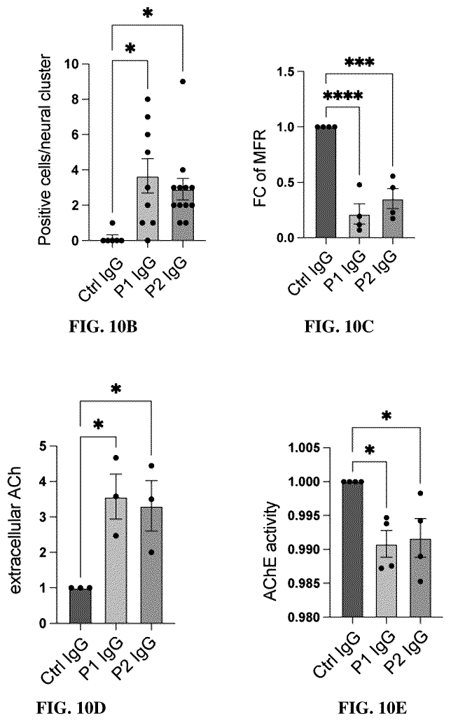

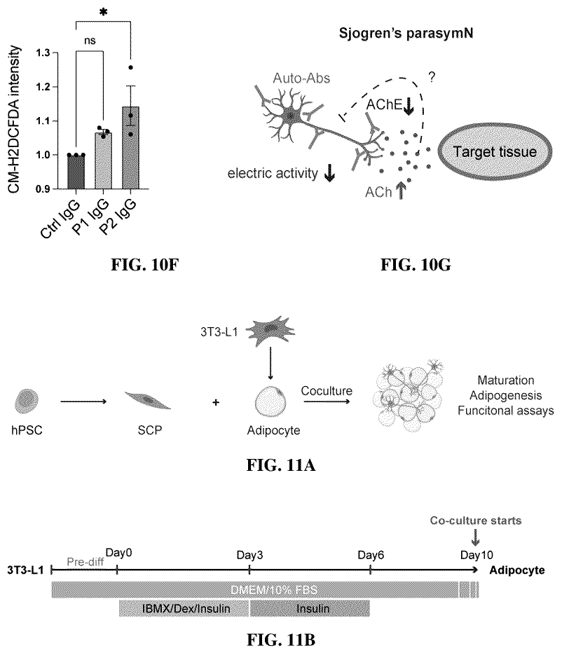

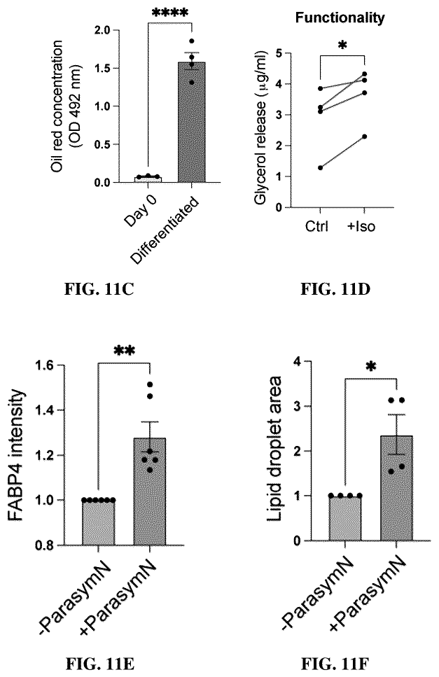

For FIG. 5B, parasymN culture medium was collected after 24 hours. For the antibody-based complement-dependent cytotoxicity assay (FIG. 10D), cell culture medium was collected at the end of the assay. Medium samples were used directly for the assay or stored at −80° C. for long-term storage. ACh level in the medium was measured using the Choline/Acetylcholine Quantification Kit (Sigma, MAK056) according to manufacturer's instruction.

Adipocyte Differentiation

3T3-L1 were seeded at 20×10³ cells/cm² and fed until 80% confluency, which was defined as day 0. From day 0-3, cells were fed with the differentiation medium containing DMEM (Gibco, 11965118), 10% FBS, 0.5 mM IBMX (Cayman, 13347), 1 μM dexamethasone (Sigma, D2915), and 10 μg/ml insulin (Sigma, 19278). From day 3-6, cells were fed with post-differentiation medium containing DMEM, 10% FBS, and 10 μg/ml insulin. After day 6, cells were maintained in DMEM with 10% FBS until the desired timepoints.

Oil Red Staining

Cells were fixed by 4% paraformaldehyde for 20 minutes and washed twice with PBS. Fixed cells were then incubated with 60% isopropanol at room temperature for 5 minutes. After removing the isopropanol, cells were air dried at room temperature for 1-5 minutes, and incubated with the Oil Red solution (Sigma, 01391) at room temperature for 10-15 minutes. After staining, cells were washed with PBS for 2-5 times until no extra Oil Red was rinsed out. For Oil Red concentration measurement, Oil Red inside the cells was extracted by 100% isopropanol for 15 minutes and measured by ELISA at 492 nm.

Lipolysis Assay

To begin with, adipocyte culture medium was replaced with prewarmed lipolysis medium containing 0.5% BSA in DMEM, and cells were incubated for 4 hours at 37° C. After 4 hours, the lipolysis medium was replaced with fresh prewarmed lipolysis medium with test drugs and incubate for 90 minutes at 37° C. Then, the cell culture medium was collected for the assay or stored at −20° C. for long-term storage. Glycerol release in adipocyte cultured medium was measured using Adipolysis Assay Kit (Cayman, 10009381) according to manufacturer's instruction.

Glucose Measurement

Medium samples from the lipolysis assay were used for glucose measurement. Glucose levels in the medium were measured and recorded by the glucose meter (Roche, ultra 2) according to manufacturer's instruction.

Statistical Analysis

Data was collected from at least (or more) three independent experiments (biological replicates), with multiple technical replicates each. Biological replicates (Chan, J. W. & Teo, A. K. K. Stem Cells 38, 1055-1059 (2020)) are defined as independent experiments conducted several days apart either started from a new frozen vial of that particular cell line (Wong, K. G., et al. Stem Cell Reports 9, 355-365 (2017)), or started from a consecutive passage number of that cell line. Multiple clones derived from one patient line have been analyzed previously (Zeltner, N., et al. Nat Med 22, 1421-1427 (2016)) and they were shown to not have significant variability, thus here one clone per iPSC line was used. Data is shown as mean±SEM. Statistical analysis is described in figures. All the analyses and graphs were processed using Prism 9.

Ethics Statement for Human Pluripotent Stem Cells

This study employed human embryonic stem cell lines (WA09), the use of which was approved to the Zeltner lab by WiCell. All iPSCs employed in this work were reprogrammed from human samples obtained through the public repository Coriell Research Institute.

Results

Schwann cell progenitor-derived parasympathetic neurons Neural crest cells (NCCs) are SOX10⁺ early embryonic progenitor cells that are highly migratory and give rise to a large variety of cell types, including all peripheral nervous system cells, melanocytes, and peripheral glia (Bronner, M. E. & Simoes-Costa, M. Curr Top Dev Biol 116, 115-134 (2016)). An efficient and defined NC differentiation protocol (Tchieu, J., et al. Cell Stem Cell 21, 399-410 e397 (2017); Fattahi, F., et al., Nature 531, 105-109 (2016)) was shown to give rise to sensory (Saito-Diaz, K., Street, J. R., Ulrichs, H. & Zeltner, N. Stem Cell Reports 16, 446-457 (2021)), enteric, and symNs (Wu, H. F. & Zeltner, N. J Vis Exp (2020); Zeltner, N., et al., Nat Med 22, 1421-1427 (2016); Saito-Diaz, K., Wu, H. F. & Zeltner, N. Curr Protoc Stem Cell Biol 49, e78 (2019)) (FIG. 1). Majd et al., employed these NCCs to derive Schwann cells (SCs), that showed identities that are similar to primary SCs and were capable of myelination (Majd, H., et al., 2022.2008.2016.504209 (2022)).

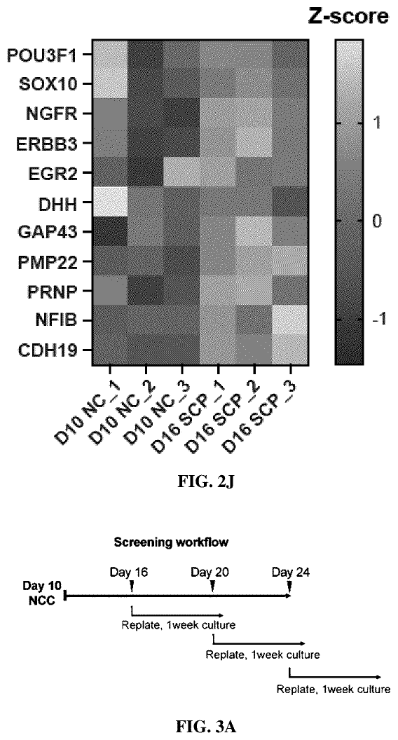

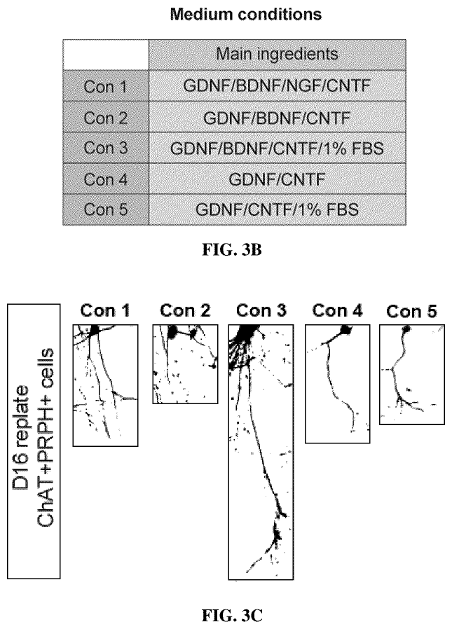

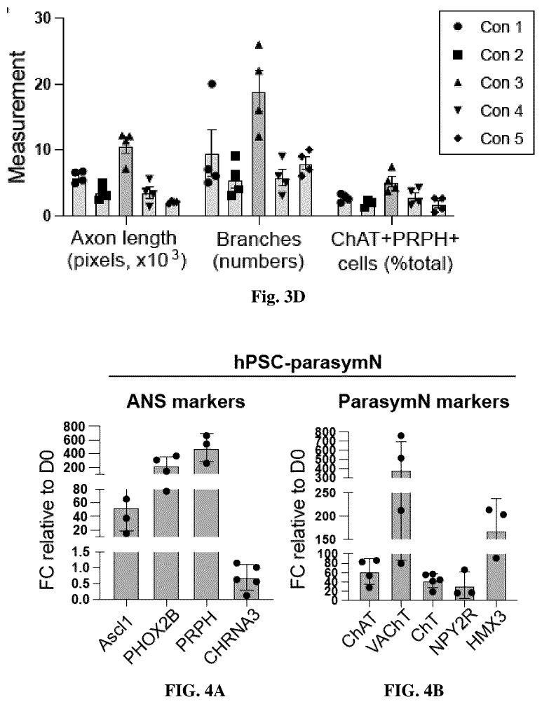

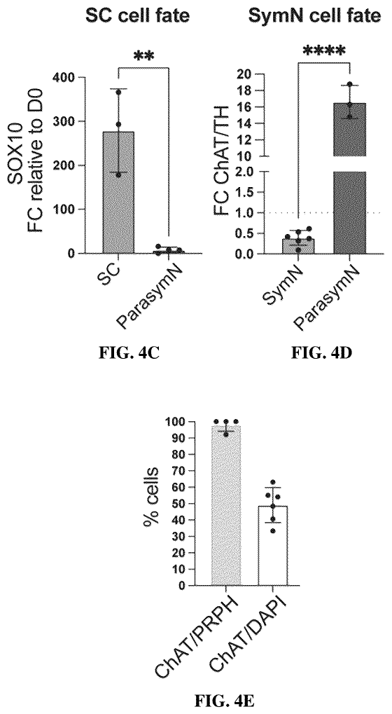

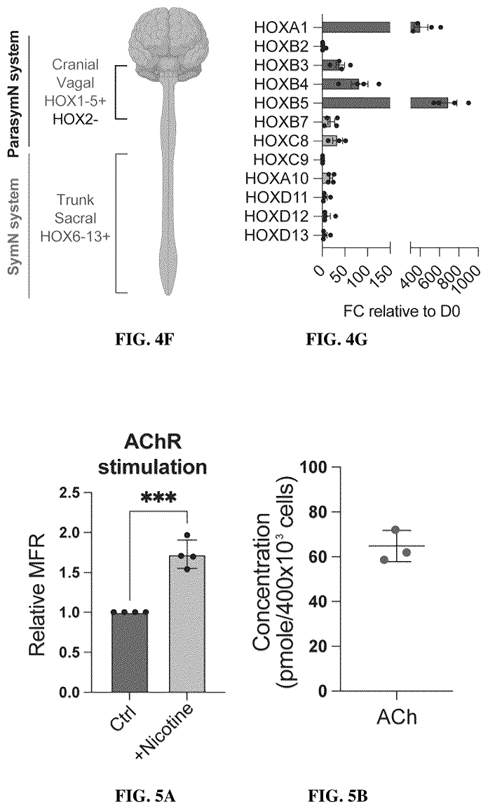

A strategy was designed to redirect differentiating SCPs into parasymNs (FIG. 1). After adaptations of the NCC phase, which increased efficiency and reproducibility, it was first aimed at capturing the time point when multipotent SCPs are induced, but not yet determined towards the SC fate. The SC protocol described a wide window of SCP existence (day 10-30, FIG. 2A). Gene expression was analyzed within this time frame of SCPs (PAX3/GAP43), immature SCs (iSC, CD56), and differentiated SCs (MPZ, S100β) using WA09 (H9) hESCs. It was observed that SCP genes decreased from day 16-20, accompanied by increasing iSC marker, which dropped after day 24, while SC genes were still increasing (FIGS. 2B-2F). These results demonstrate a clear progress of SC differentiation and indicate that the proper timing to start parasymN induction may be between day 16-24. To differentiate SCPs into parasymNs, a timepoint and factor screen were performed, using replating every four days between day 16 and 24, and GDNF, BDNF, NGF, FBS and CNTF combinations (FIGS. 3A-3B). Ciliary neurotrophic factor (CNTF) is important for supporting parasymN development and survival and has been shown as the key trophic factor for parasymNs in target tissue innervation and communication (Helfand, S. L., Smith, G. A. & Wessells, N. K. Dev Biol 50, 541-547 (1976); Wang, X. & Halvorsen, S. W. J Neurosci 18, 7372-7380 (1998)). One week after replating, it was noted that only replating before day 16 induced neuron-like cell morphology, and cells grown in condition 3 (GDNF, BDNF, CNTF, 1% FBS) displayed well developed neurite bundles. This was further confirmed by staining day 16 replated cells with all medium conditions for peripheral neuron marker PRPH and the parasymN neurotransmitter choline acetyltransferase (ChAT). Thus, it was concluded that day 16 replating in condition 3 medium is ideal for parasymN differentiation. Upon maturation to day 30, parasymNs expressed autonomic markers ASCL1, PHOX2B, PRPH, and CHRNA3 by RT-qPCR (FIGS. 4A-4B), possibly ruling contamination of other peripheral cholinergic neural types, such as enteric and sensory neurons. They were also positive for parasympathetic (which is mainly cholinergic) markers ChAT, VAChT, ChT, and NPY2R (FIGS. 4A-4B). To check if there are remaining SCs after differentiation, SOX/0 expression of day 30 hPSC-parasymNs was compared to original day 30 hPSC-SCs and it was noted that the differentiated neurons in this study barely expressed SOX/0 (FIGS. 4C-4D), indicating high differentiation specificity. The ratio of ChAT (cholinergic) to TH (adrenergic) expression between day 30 hPSC-parasymNs and hPSC-symNs (Wu, H. F. & Zeltner, N. J Vis Exp (2020)) was also compared and it showed that hPSC-parasymNs predominantly expressed ChAT, while hPSC-symNs expressed TH (FIGS. 4C-4D). This was also confirmed at protein level using western blot. The expression of ChAT, VAChT, and NPY2R in hPSC-parasymNs were then confirmed by immunostaining, and PHOX2B expression in the neurons were also shown using an PHOX2B::GFP H9 reporter line (Oh, Y., et al., Cell Stem Cell 19, 95-106 (2016)). Quantifying the images showed that the differentiation efficiency of this protocol yielded about 50% ChAT parasymNs with, however, very high differentiation specificity as almost all PRPH neurons expressed ChAT (FIG. 4E). To further validate the identity of hPSC-parasymN, the expression profile of HOX genes was investigated. In human ANS, parasymN chain are derived mainly from cranial and vagal nerves, which are HOX 1-5⁺, but HOX2⁻ since it represents cranial nerve V that gives rise to trigeminal and sensory motors, but not to parasymNs (Mendez-Maldonado, K., Vega-Lopez, G. A., Aybar, M. J. & Velasco, I. Front Cell Dev Biot 8, 635 (2020)) (FIG. 4F). RT-qPCR analysis showed that the hPSC-parasymNs differentiated in this study mainly expressed HOX 1-5, except HOX 2 (FIG. 4G). It seemed that hPSC-parasymNs in this study covered multiple regions of the parasymN chain. The cranial nerve III-derived parasymN ganglion is HOX null. There is no specific marker to identify parasymN with cranial origin. Based on the variety of genes detected in hPSC-parasymNs, hPSC-parasymNs display high differentiation specificity, robust parasymN identity, and cover most parasymNs types in the body.

Next, whether the hPSC-parasymNs can functionally recapitulate parasymN characteristics was examined. Using microelectrode array (MEA), spontaneous action potential was detected in day 30 hPSC-parasymNs. The activity was further inducible by stimulating the cholinergic receptors by nicotine (FIG. 5A). The release of ACh, the main neurotransmitter of parasymN, was also detected in the culture medium by ELISA (FIG. 5B). While symNs express adrenergic receptors to regulate NE secretion, parasymNs mainly express cholinergic muscarinic receptors (MusR) (Wehrwein, E. A., Orer, H. S. & Barman, S. M. Compr Physiol 6, 1239-1278 (2016)). Thus, the expression of all MusR subtypes was assessed in hPSC-parasymNs and it was found that the M2 and M4 MusRs were the major type of MusR in hPSC-parasymNs (FIG. 5C), consistent with findings in human and animal parasymN tissues (Wehrwein, E. A., Orer, H. S. & Barman, S. M. Compr Physiol 6, 1239-1278 (2016); Fryer, A. D. & Jacoby, D. B. Am J Respir Crit Care Med 158, S154-160 (1998); Oberhauser, V., Schwertfeger, E., Rutz, T., Beyersdorf, F. & Rump, L. C. Circulation 103, 1638-1643 (2001)). Based on this, hPSC-parasymNs were treated with bethanechol (BeCh) and atropine, clinical MusR agonist and antagonist, respectively (Fryer, A. D., et al. Am J Respir Cell Mol Biol 15, 716-725 (1996); Konopka, L. M. & Parsons, R. L. Neuropharmacology 31, 1311-1321 (1992); Suaid, H. J., Rocha, J. N., Martins, A. C., Cologna, A. J. & Tucci, S. J. Int Braz J Urol 29, 162-165 (2003)). It was observed that BeCh activated, while atropine inactivated hPSC-parasymNs (FIGS. 5D-5E). Furthermore, upon treatment with 6-OHDA (100 11M), a neurotoxin that mainly targets catecholaminergic neurons (i.e. symNs) and can trigger apoptosis in 12 hours, hPSC-symNs displayed early hyperactivity, an implication of neurodegeneration (Takayama, Y., et al. Sci Rep 10, 9464 (2020); Fujita, H., et al. Brain Res 1113, 10-23 (2006); Han, B. S., Noh, J. S., Gwag, B. J. & Oh, Y. J. Neurosci Lett 341, 99-102 (2003); Lee-Liu, D. & Gonzalez-Billault, C. J Neurochem 158, 586-588 (2021)). In contrast, hPSC-parasymNs remained unaffected (FIG. 5F). To test whether hPSC-parasymNs can build functional connectivity to the target tissues, a co-culture system using hPSC-parasymNs and hPSC-cardiomyocytes (CMs) was established. During development, SCPs migrate along the axons of preganglionic neurons to the locations close to the target tissues and differentiate into parasymNs. To rebuild this developmental process in vitro, day 16 hPSC-SCPs were replated on top of hPSC-CMs (Lin, Y. & Zou, J. STAR Protoc 1 (2020)) (FIG. 5G). 10 days after co-culture, TUJ1⁺ hPSC-parasymNs targeting α-ACTININ+ CMs were identified using immunofluorescence imaging, which was evident by the formation of neurocardiac junctions (NCJs) observed as nodal structures (Zaglia, T. & Mongillo, M. J Physiol 595, 3919-3930 (2017)). To confirm the functional connectivity, the parasymNs were activated in the co-cultures with nicotine and measured the beating rate of hPSC-CMs. It was noted that the beating of hPSC-CMs decreased after treatment with nicotine (FIG. 5H). These data indicate that these hPSC-parasymNs are fully functional.

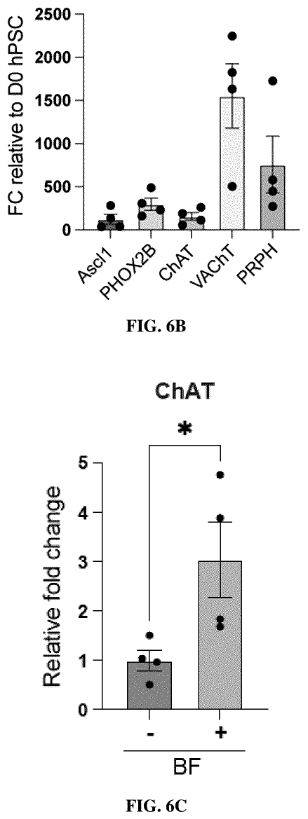

Next, it was tested if this protocol can be performed with different hPSC lines. ParasymNs were differentiated with another hESC line (MEL1, male) and four hiPSC lines (healthy control 652, 11 y.o. female, Cpl, 21 y.o. female, hDFn, male, neonate and C1, 35 y.o. female) (Zeltner, N., et al., Nat Med 22, 1421-1427 (2016)). ParasymNs from all lines were positive for ChAT and PRPH as determined by immunofluorescence imaging, with similar efficiencies (FIG. 6A). It has also been showed that a commercial neural supplement BrainFast facilitated parasymN differentiation as more neurite bundles were observed at an earlier stage. It was tested whether the instant differentiation strategy works on SCPs from other SC protocols. Kim et al. published a hPSC-SC protocol in 2017, although the SCs from this protocol did not display efficient functional myelination (Kim, H. S., et al. Stem Cell Reports 8, 1714-1726 (2017)). Then the current hPSC-parasymN protocol was applied to this SC protocol in the first week of SCP induction and successfully differentiated neurons that were positive for ChAT, PRPH and other parasymN markers (FIG. 6B). Taken together, the current hPSC-parasymN protocol is highly specific, flexible, reproducible, functional, and consistent.

### Example 2: Parasympathetic Neurons Derived from Human Pluripotent Stem Cells Model Human Diseases and Development

Parasympathetic neurons (parasymNs) are important for unconscious body responses, including rest-and-digest and calming the body. ParasymN dysfunction plays important roles in diseases such as autonomic neuropathy; autoimmune disease may attack parasymNs; and parasymN innervation is important for organ development. However, human parasymN function and dysfunction is vastly understudied, due to the lack of a model system. Human pluripotent stem cell (hPSC)-derived neurons can fill this void and serve for disease modeling, drug screening, and transplantation therapy.

Here, a differentiation paradigm detailing has been developed based on the derivation of functional human parasymNs from Schwann cell progenitors (SCP). As described in more detail below, these neurons can be employed (i) to model neuropathy in the genetic disorder Familial Dysautonomia, (ii) to show parasymN dysfunction during SARS-CoV-2 infection, (iii) to model the autoimmune disease Sjorgen's syndrome and, (iv) to show that parasymNs innervation of white adipocytes during development and helps mature the tissue.

The model system will be instrumental for future disease mechanistic and drug discovery studies as well as for human developmental studies.

hPSC-parasymNs Reveal Parasympathetic Neuropathology in Familial Dysautonomia

With these human parasymNs in hand, it was sought to model diseases with parasympathetic neuropathy, especially those not fully understood due to the limitation of human model systems. The first was Familial dysautonomia (FD), a genetic disease that mainly affects the ANS, causing abnormalities in controlling body temperature, gland secretion, and blood pressure (Norcliffe-Kaufmann, L., Slaugenhaupt, S. A. & Kaufmann, H. Prog Neurobiol 152, 131-148 (2017)). One of the most life-threatening symptoms in FD is the dysautonomic crisis. Induced by stress, FD patients display extreme cardiovascular dysregulation, hypertension, high blood pressure, vomiting attacks, and diffused sweating. Although both SNS and PSNS are found affected in FD patients, the level of involvement and the defects in parasymNs remain understudied (Dutsch, M., et al. J Neurol Sci 195, 77-83 (2002); Stemper, B., et al. Neurology 63, 1427-1431 (2004)). Both hypoactivity and hyperactivity of FD PSNS have been suggested from clinical observations (Bremner, F. D. & Smith, S. E. J Neuroophthalmol 26, 209-219 (2006); Hilz, M. J., et al. Am J Physiol 276, R1833-1839 (1999); Maayan, C., Katz, E., Begin, M., Yuvchev, I. & Kharasch, V. S. Clin Pediatr (Phila) 54, 174-178 (2015)). A recent study showed that FD symNs are spontaneously hyperactive and provided the underlying mechanisms using the hPSC-symN platform (DOI: doi.org/10.21203/rs.3.rs-1024649/v1).

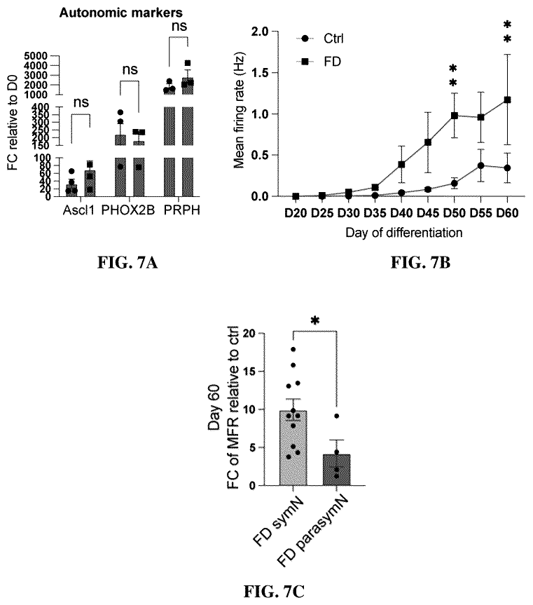

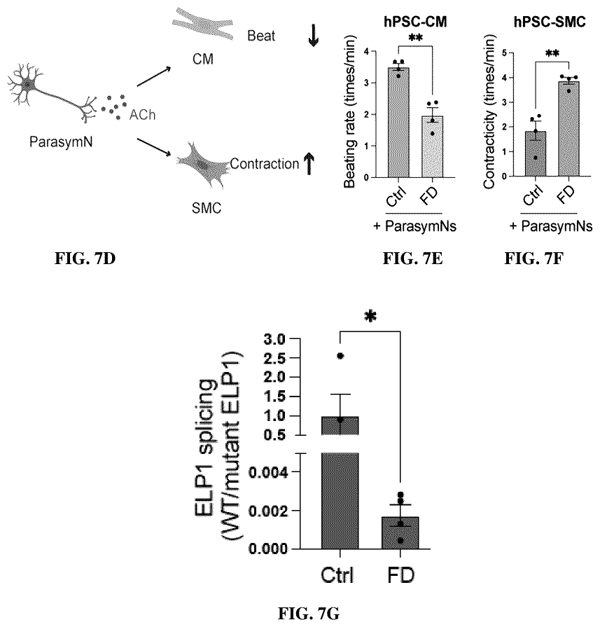

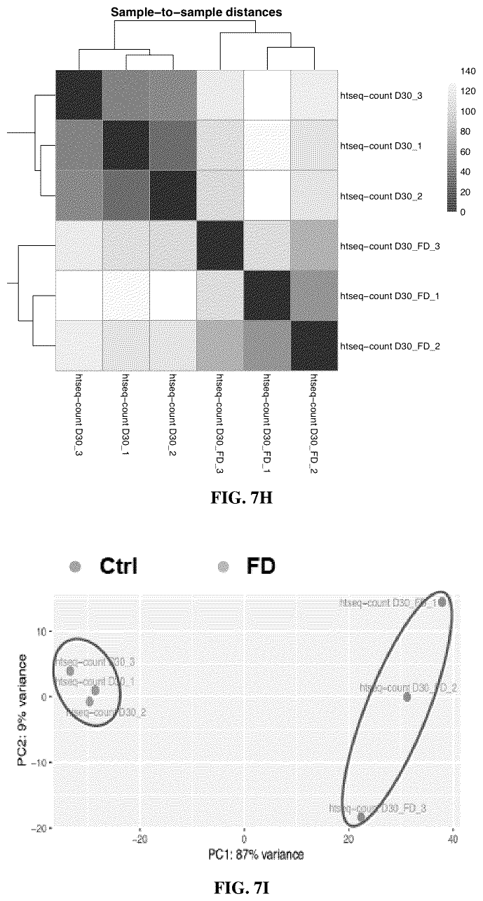

Here, it was set out to better understand the FD parasymN pathology and its interaction with symNs. Thus, parasymNs were differentiated from control (hPSC-ctrl-H9 ESC and iPSC-ctrl-C1, hereafter data from these lines was combined) and FD (iPSC-FD-S2). FD parasymNs on day 30 showed similar neural morphology as control parasymNs using immunofluorescence staining of PRPH and their gene expression was not significantly different (FIG. 7A). MEA analysis showed that FD parasymNs were hyperactive compared to control (FIG. 7B), but the level of parasymN hyperactivity was not as high as symN hyperactivity in FD (FIG. 7C). These results mimic parasymN phenotypes in FD patients, which are subtler than symN phenotypes.

To test if hyperactive FD parasymNs affect different target tissues, two co-culture systems were preformed with hPSC-CMs and hPSC-smooth muscle cells (Fattahi, F., et al. Nature 531, 105-109 (2016); Patsch, C., et al. Nat Cell Biol 17, 994-1003 (2015)) (SMCs, FIG. 7D). Generally, ACh signaling leads to decreased beating of cardiac muscles, but increased contraction of smooth muscle due to the expression of different ACh receptors (Veach, H. O. J Physiol 60, 457-478 (1925)). Differentiated parasymNs were observed after co-culturing hPSC-SCPs with hPSC-SMCs, with direct contacts to SMCs evident by synaptophysin⁺ (SYP) neuromuscular junctions. Indeed, hyperactive FD parasymNs lowered CM beating, while triggering SMC contraction compared to control parasymNs (FIGS. 7E-7F). The data confirmed a parasymN specific phenotype in FD, which may contribute to altered body homeostasis in this disease.

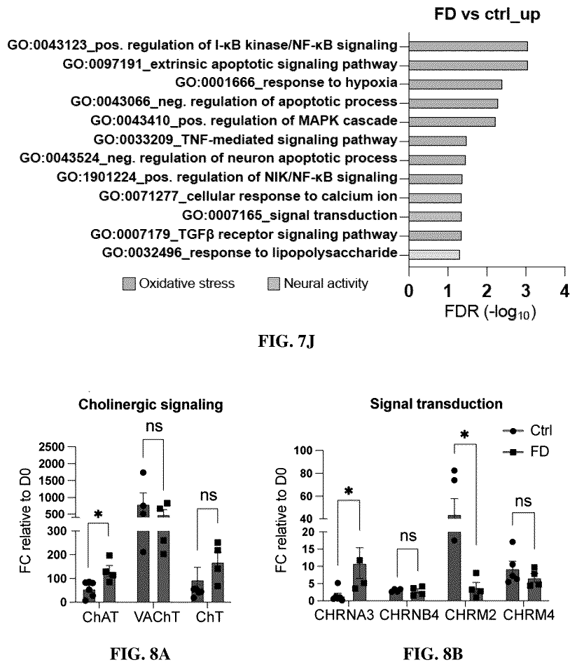

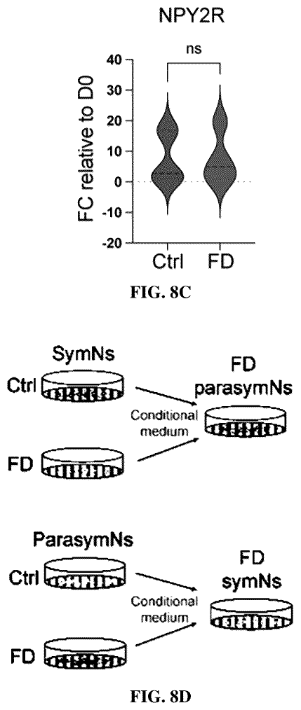

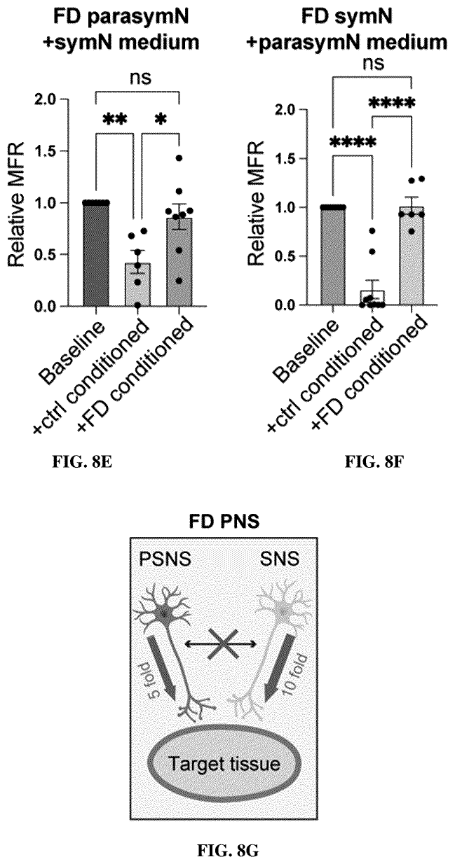

To understand the mechanisms of parasymN hyperactivity in FD better, genes that are related to the regulation of neural activity were analyzed. The cholinergic signaling marker ChAT, but not VAChT and ChT, increased in FD parasymNs, supporting the hyperactivity phenotype (FIG. 8A). When comparing markers of signaling receptors, it was noted that FD parasymNs expressed higher nicotinic receptor (CHRNA3), which receives the preganglionic signals, but lower MusR (CHRM2), which autoregulates ACh release (FIG. 8B). These findings indicate that FD parasymNs are hypersensitive to the external stimuli, but lack of the autoregulatory machinery.

With individual phenotypes found in FD parasymNs and FD symNs, how these two systems work together in FD was next investigated. Parts of the ANS, parasymNs and symNs communicate through secretory factors and specific membrane receptors. For instance, parasymNs activity can be downregulated through expressing the neuropeptide Y (NPY) receptor (NPY2R), which is targeted by NPY released from symNs as a cofactor of NE (Fedele, L. & Brand, T. J Cardiovasc Dev Dis 7(2020); Schwertfeger, E., et al. Naunyn Schmiedebergs Arch Pharmacol 369, 455-461 (2004); Tan, C. M. J., et al. Front Physiol 9, 1281 (2018)). In cardiovascular diseases, such as hypertension, oversecreted NPY from symNs inhibits parasymN activity, therefore leads to unbalanced ANS. Based on this, NPY2R levels were first compared in control and FD parasymNs, but no significant difference was found (FIG. 8C). To better evaluated the effects of parasymNs and symNs to each other in FD, reciprocal conditional medium treatments was performed. Conditional media from control and FD symNs were given to FD parasymNs, and vice versa (FIG. 8D). Interestingly, control media from parasymNs or symNs had the ability to suppress the activity of their FD counterparts, an effect that was abolished when FD conditional media were added (FIGS. 8E-8F). These findings revealed for the first time that in FD, parasymNs and symNs are both hyperactive, while symN hyperactivity is stronger than parasymN hyperactivity. The important crosstalk and mutual regulation between the two systems is impaired, which explains the general oversensitivity and overreaction of FD patients ANS. (FIG. 8G).

hPSC-parasymNs for COVID-19 Research

COVID-19 has caused significant damage to human lives. Respiratory failure and cardiovascular pathologies are the major causes of death after SARS-CoV-2 infection (South, A. M., Diz, D. I. & Chappell, M. C. COVID-19, Am J Physiol Heart Circ Physiol 318, H1084-H1090 (2020)). As the key regulator of respiratory and cardiovascular functions, SNS hyperactivation has been found in patients of COVID-19 (Porzionato, A., et al. FEBS J 287, 3681-3688 (2020); Del Rio, R., Marcus, N. J. & Inestrosa, N. C. Front Physiol 11, 561749 (2020)). Moreover, symNs also express angiotensin-converting enzyme 2 (ACE2), the membrane receptor/enzyme which is the gateway for SARS-CoV-2 invasion, indicating that symNs could be a target of SARS-CoV-2 (Bardsley, E. N., Neely, O. C. & Paterson, D. J. J Mol Cell Cardiol 138, 234-243 (2020)). Recently, a recent study has shown that the hPSC-symNs express ACE2, and that hPSC-symNs were hyperactivated upon infection with SARS-CoV-2 pseudovirus infection (Wu, H. F., et al. Clin Auton Res 32, 59-63 (2022)). However, the role of the PSNS in COVID-19 remains unclear.

ACE2 belongs to the renin-angiotensin-aldosterone system (RAAS), the key system for blood pressure and blood volume maintenance. For a healthy RAAS, the proper balance of its two arms is important. Within the RAAS, angiotensin II (Ang II) induces vasocontraction, which is counteracted by angiotensin 1-7 that is hydrolyzed from Ang II by ACE2. Unbalanced RAAS, due to compromised ACE2 by the binding of SARS-CoV-2 is a major cause of multiple organ damage in patients (Sharma, R. K., et al. Hypertension 76, 651-661 (2020)).

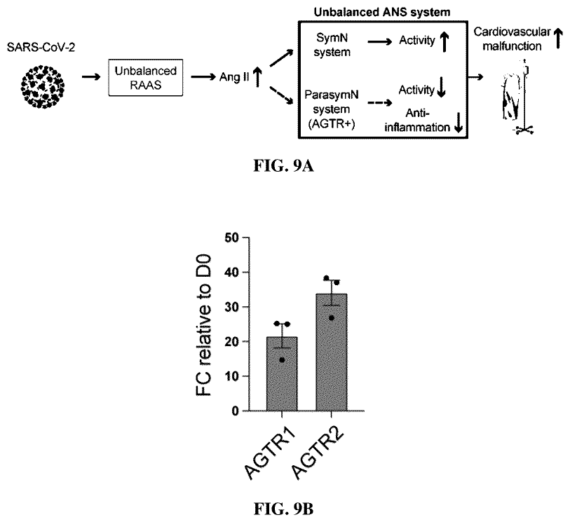

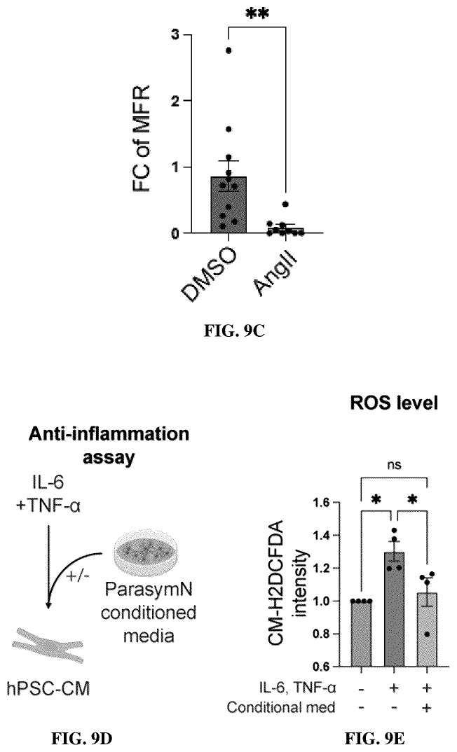

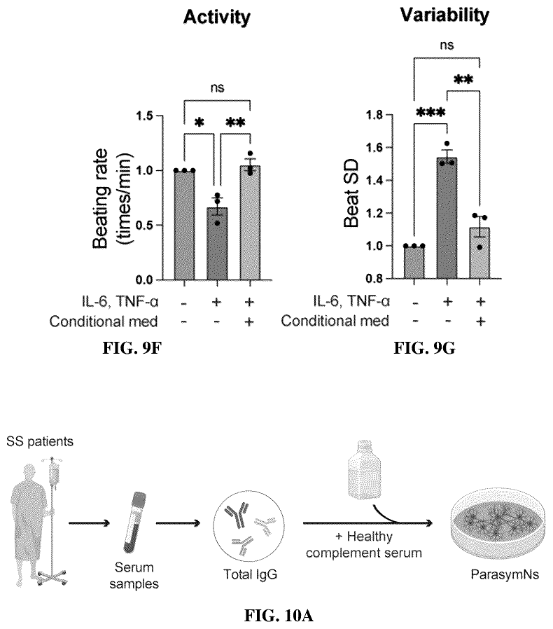

PSNS may be a key player in COVID-19-related illness for the following reasons. First, because human parasymNs express angiotensin receptors, the target of Ang II, parasymNs could be sensitive to changes caused by an unbalanced RAAS system (Shanks, J. & Ramchandra, R. Int J Mol Sci 22(2021)) (FIG. 9A). Indeed, increased Ang II level may decrease the activity of PSNS, which worsens the unbalanced ANS, since symNs are hyperactivated. Second, the anti-inflammatory effect mediated by ACh is one of the important regulators of inflammation in the PNS (Udit, S., Blake, K. & Chiu, I. M. Nat Rev Neurosci 23, 157-171 (2022)). Decreased parasymN activity during SARS-CoV-2 infection therefore may not only cause unbalanced ANS, but also weaken the defense system in the body (FIG. 9A).

Experiments were designed to evaluate whether the hPSC-parasymNs can be used to study COVID-19 related parasymN responses. Expression of Ang II receptors, AGTR1/2, in hPSC-parasymNs were analyzed and confirmed (FIG. 9B). Treating hPSC-parasymNs with Ang II decreased parasymN activity (FIG. 9C), indicating that hPSC-parasymNs are functionally responsive to COVID-19-related homeostatic changes. Next, experiments were designed to assess the anti-inflammatory functionality of hPSC-parasymNs in vitro. Recently, Yang et al. used a hPSC-CM and hPSC-macrophage co-culture system to show that TNF-α and IL-6 released by immune cells are one of the main mechanisms of cardiovascular complications in COVID-19 (Yang, L., et al. Circ Res 129, 33-46 (2021)). Based on this, an anti-inflammation assay was performed in which hPSC-CMs were challenged by TNF-α and IL-6, with or without conditional media from hPSC-parasymNs (FIG. 9D). 24 hours after treatment, ROS level of CMs was analyzed using the CM-H2DCFDA oxidative stress indicator (Oparka, M., et al. Methods 109, 3-11 (2016)). Consistent with previous research, TNF-α and IL-6 significantly increased the ROS level in CMs (FIG. 9E). However, hPSC-parasymN-conditioned media ameliorated the ROS accumulation. To check if parasymN media can improve CM functionality under the inflammatory factors, cardiac activity of CMs was measured using MEA. TNF-α and IL-6 decreased the beating rate yet increased the beating variability of CMs (FIGS. 9F-9G), indicating that CM functions are affected in the inflammatory state. With the addition of parasymN media, both CM beating and beating variability were significantly restored to the levels that were similar to healthy CMs. These results demonstrate significant parasymN involvement in COIVD-19 disease and will be an important tool to further research and possible treatment options in the future.

hPSC-parasymNs Model Sjogren's Syndrome

Next parasymN specific phenotypes were studied in Sjogren's syndrome (SS), a female-predominant autoimmune disease affecting ˜3 million people in the US, that is often a precursor to more deleterious autoimmune disorders (Manfre, V., et al. Clin Exp Rheumatol 38 Suppl 126, 10-22 (2020)). SS patients suffer from difficulties in producing tears and saliva, which are controlled by the ANS (Davies, K. & Ng, W. F. Front Immunol 12, 702505 (2021)). They are 24 times more likely to develop B-Cell lymphoma and other complications, such as vasculitis. There is no FDA-approved therapy available for this disease. Current clinical characteristics have defined SS as primary SS (pSS) and secondary SS (sSS), where patients experience primarily autoimmune symptoms or those are accompanied by rheumatic diseases, respectively. Accordingly, pSS patients usually exhibit high levels of anti-SSA/Ro and/or anti-SSB/La antibodies (Mavragani, C. P. & Moutsopoulos, H. M. J Rheumatol 46, 665-666 (2019)). Years of case studies have indicated the involvement of parasympathetic neuropathy in SS, especially pSS. For instance, autoantibodies targeting MusRs are found in pSS patients, which impairs ACh-mediated signal transduction (Li, J., et al. Lab Invest 84, 1430-1438 (2004)). Another common symptom in SS patients is prolonged fatigue, which may be caused by PSNS hypoactivation (Brunetta, E., et al. Front Physiol 10, 1104 (2019)). In FD, as a result of ANS neuropathy, dry eyes is also a common feature (Dietrich, P. & Dragatsis, I. Genet Mol Biol 39, 497-514 (2016)). Recent findings of symN hyperactivity in FD (DOI: https://doi.org/10.21203/rs.3.rs-1024649/v1) lead to the question whether intrinsic parasymN hypoactivity in addition to the blockage of downstream signaling due to the anti-MusRs antibodies is underlying clinical symptoms in SS patients. Indeed, vagal nerve stimulation has been shown to improve the symptoms in pSS patients. ParasymNs express MusR (FIG. 5C), which may make the PSNS itself a target of at least one of the identified autoantibodies in pSS.

Therefore, here it was aimed to model and evaluate the parasymN response in SS. To do so, an antibody-based complement-dependent cytotoxicity assay was performed to model SS in vitro. Total IgG was purified from sera from two SS patients, one with reported PNS involvement and another who has high anti-SSA. Total IgG was added to healthy hPSC-parasymNs together with complement-active healthy human serum (FIG. 10A). Healthy total human IgG was used as the control. After 72 hour, hPSC-parasymNs were targeted by autoantibodies from both SS patients (FIG. 10B), indicating that parasymNs might be a direct target of autoimmune response. Next parasymN activity compared after treatment with SS patient IgG. ParasymN activity dropped upon treatments with SS IgGs compared to control, indicating that direct parasympathetic dysfunction might partially contribute to the symptoms of Sjogren's syndrome (FIG. 10C). Accordingly, a possible projected result was to find decreased extracellular ACh levels in parasymN media. Interestingly, upon SS IgG treatment of parasymNs, an increase was found compared to control-IgG treated neurons (FIG. 10D). Thus expression of ACh synthesis, ACh signaling, and neural plasticity-related genes were analyzed, but none of them was changed significantly after treatment with SS IgG. Another pathway to increase ACh in the synapse is to suppress ACh degradation, which in parasymNs occurs via acetylcholinesterase (AChE). It was found that the activity of AChE was significantly decreased in SS-IgG treated parasymN media (FIG. 10E). In addition, ROS levels were elevated in one SS patient (P1) IgG-treated parasymNs and significantly increased in another (P2, FIG. 10F). Together, these findings indicate that parasymN function is directly affected in SS patients, and that the increased ACh level is a result of inhibited AChE in response to the autoimmune response (FIG. 10G).

hPSC-parasymNs Target White Adipocytes In Vitro

In addition to disease modeling, experiments were designed to investigate current biological conundrums using hPSC-parasymNs. It has been recognized that the SNS plays important roles in lipid metabolism. SymNs target all types of adipose tissues, including white, brown, and beige adipocytes (Bartness, T. J. & Ryu, V. Int J Obes Suppl 5, S35-39 (2015)). Activation of symNs stimulates lipolysis and increases glucose uptake in white adipose tissues (WAT) (Blaszkiewicz, M., Willows, J. W., Johnson, C. P. & Townsend, K. L. Biology (Basel) 8(2019)). A functional co-culture model of symNs and white adipocytes has been established using primary symNs with 3T3-L1 mouse preadipocyte-derived white adipocytes, a widely used in vitro model system to study WAT (Turtzo, L. C., Marx, R. & Lane, M. D. Proc Natl Acad Sci USA 98, 12385-12390 (2001)). The role of the PSNS in WAT, in contrast, is not fully understood and somewhat controversial. Studies showing that parasymNs do or do not innervate WAT were proposed (Kreier, F., et al. J Clin Invest 110, 1243-1250 (2002); Bartness, T. J. J Clin Invest 110, 1235-1237 (2002); Kreier, F. & Buijs, R. M. Am J Physiol Regul Integr Comp Physiol 293, R548-549; author reply R550-542, discussion R553-544 (2007); Giordano, A., et al. Am J Physiol Regul Integr Comp Physiol 291, R1243-1255 (2006)). Most importantly, none of the attempts to dissect parasympathetic innervation to adipose tissues are based on human parasymN models.

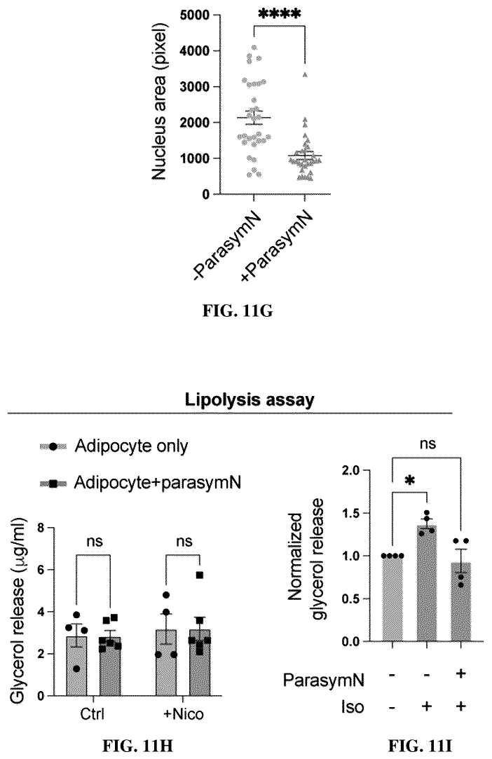

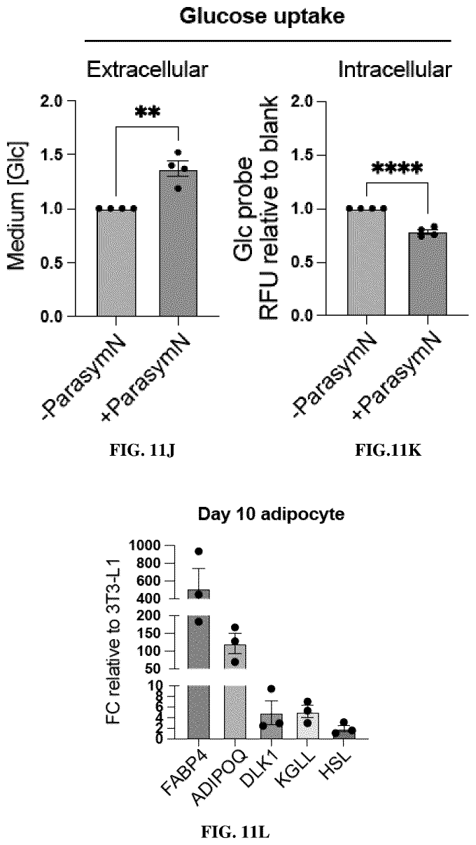

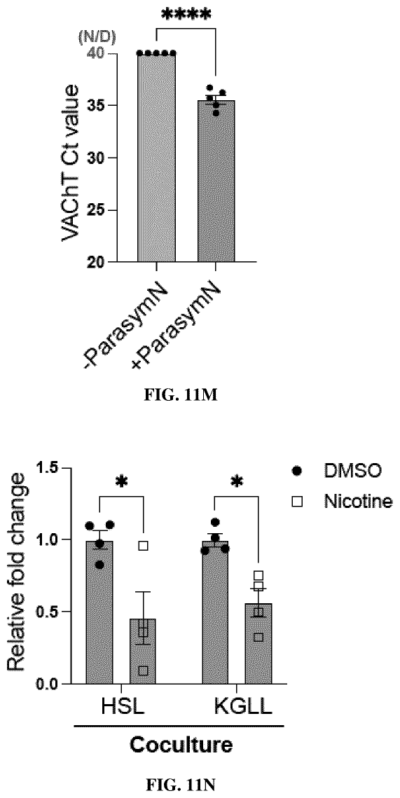

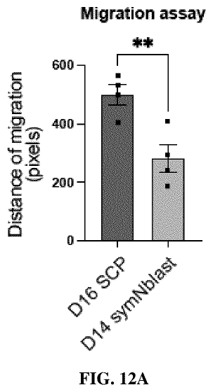

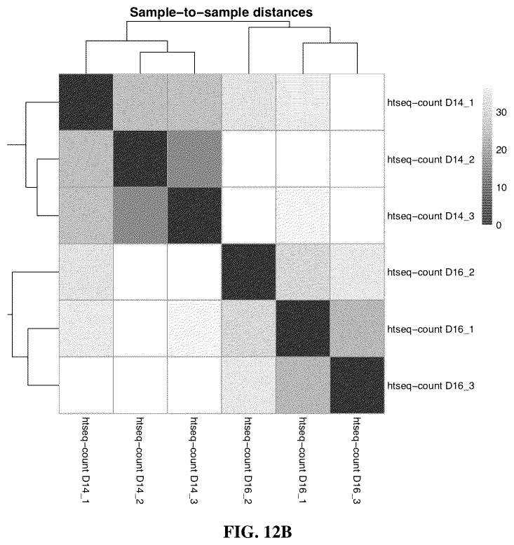

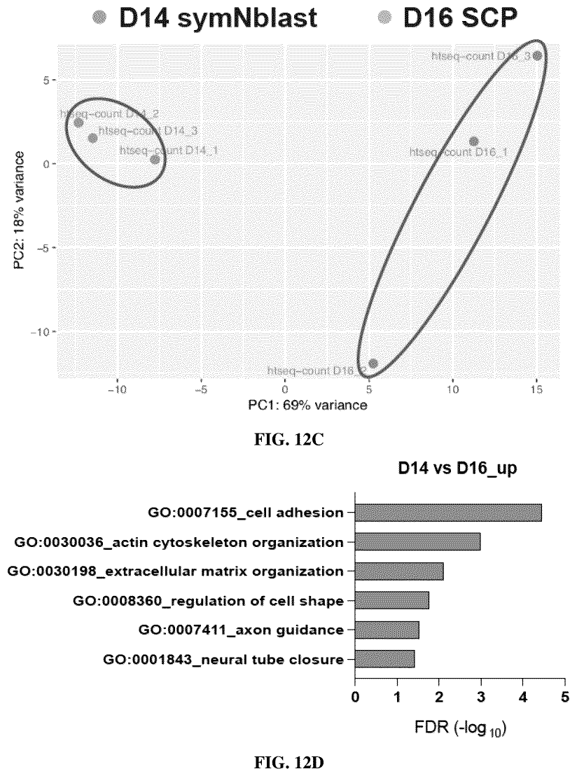

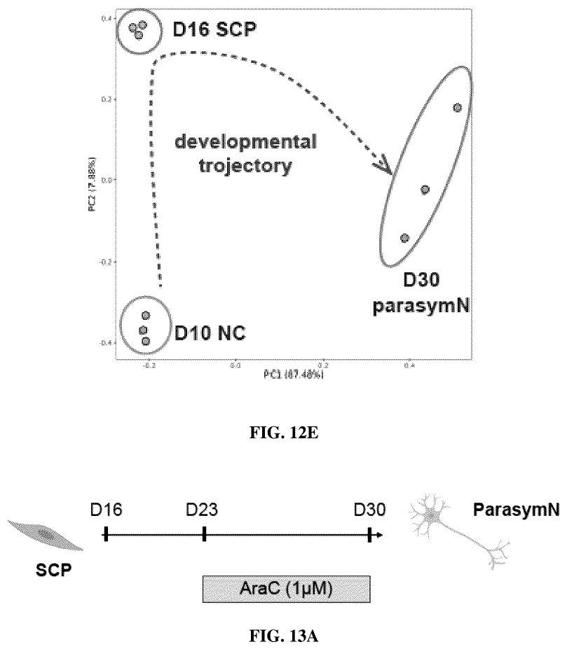

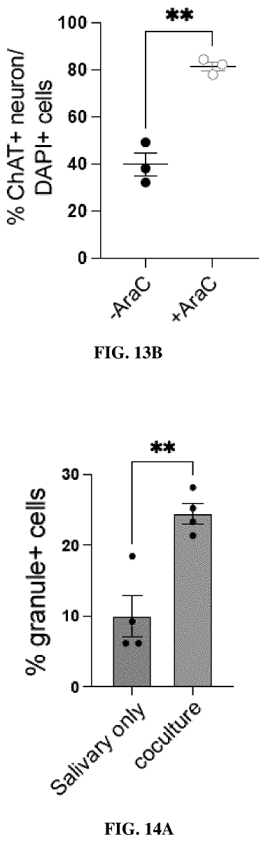

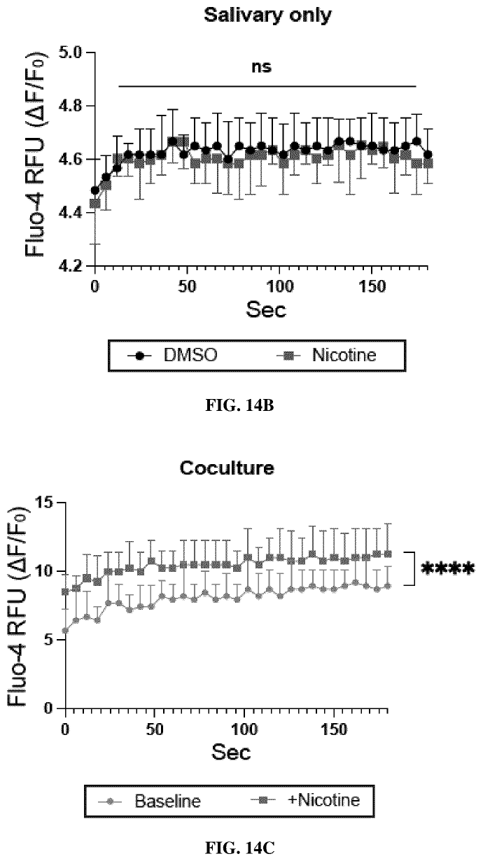

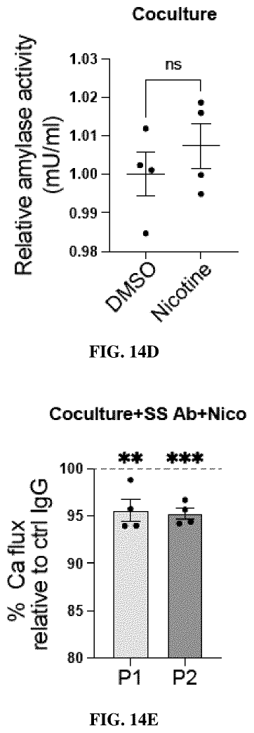

Thus, it was tested whether hPSC-parasymNs can innervate WAT and/or regulate WAT metabolism. Day 16 SCPs were co-cultured with 3T3-L1-derived white adipocytes on day 10 (FIGS. 11A-11B). Differentiated adipocytes alone showed accumulating lipid droplets (FIG. 11C) and displayed lipolytic activity under the treatment of β-adrenergic receptor agonist isoproterenol (FIG. 11D). 10 days after co-culture, parasymNs were observed on FABP4⁺ adipocytes, and the direct contact was confirmed by SYP staining using immunofluorescence imaging. This result indicates that hPSC-parasymNs innervate white adipocytes in vitro. Next the effects of parasymN innervation on adipocyte maturation were examined. First, as an adipocyte maturation marker, FABP4 expression was significantly increased in adipocytes co-cultured with parasymNs (FIG. 11E). The level of adipogenesis was examined using the lipid droplet dye LipidSpot and found that adipocytes co-cultured with parasymNs displayed higher adipogenesis activity (FIG. 11F). Moreover, it has been shown that the size of nuclei decreases in mature adipocytes (McColloch, A., Rabiei, M., Rabbani, P., Bowling, A. & Cho, M. Sci Rep 9, 16381 (2019)). Indeed, smaller nuclei were found in co-cultured adipocytes compared adipocyte cultured alone (FIG. 11G). Together, it has been shown that parasymNs promote white adipocyte maturation in vitro. To test whether adipose functions can be regulated by parasymN activity, the lipolytic activity was compared. It was noted that either in adipocytes only or in the parasymN co-cultured condition, lipolysis levels remained unchanged, even when parasymNs were stimulated by nicotine (FIG. 11H). However, parasymNs counteracted the effect on increasing lipolytic levels induced by isoproterenol (FIG. 11I), indicating that parasymNs negatively regulate WAT lipolysis. In addition, co-culturing adipocytes with parasymNs increased medium glucose levels, indicating decreased glucose uptake in adipocytes (FIG. 11J). Taken together, the results demonstrate that human parasymNs target WAT, and regulate WAT maturation and functionality in vitro.

### SUMMARY

In this study, the first postganglionic parasymN differentiation strategy has been developed from hPSCs where the cells pass appropriately through the SCP stage. hPSC-parasymNs exhibit parasymN identity and functionality and can be co-cultured with various cell types to show functionality. A previous study has established an autonomic progenitor cell differentiation protocol and found that both sympathetic-like (adrenergic) and parasympathetic-like (cholinergic) neurons can be induced from the same progenitors. However, considering that symNs and parasymNs have diverse developmental origins, NCCs and SCPs, respectively, and that certain symNs are cholinergic (Ernsberger, U. & Rohrer, H. Neural Dev 13, 20 (2018)), the cholinergic neurons generated from this protocol might not be true parasymNs. In this protocol, the parasymN differentiation medium contains no NGF. Given that NGF has been shown important for symN survival (Chun, L. L. & Patterson, P. H. J Cell Biol 75, 712-718 (1977)), the current method may exclude potential symN contamination from not fully differentiated NCCs. Moreover, during embryonic development, early differentiating parasymNs and symNs express both adrenergic/cholinergic genes until the postmitotic stage. The ChAT/TH ratio (FIG. 4D) therefore not just shows low symN contamination, but also supports that these hPSC-parasymNs are already at an early stage of maturation.

It has been shown that the PSNS in FD is defective in addition to the SNS. However, onset and extent of parasympathetic phenotypes in FD may be later and subtler than the sympathetic phenotypes. Using indirect cardiovascular measurements, such as the cold face test, heart rate, blood pressure, and respiratory time, clinical studies have indicated that lower parasympathetic drive may be underlying FD pathologies (Carroll, M. S., Kenny, A. S., Patwari, P. P., Ramirez, J. M. & Weese-Mayer, D. E. Pediatr Pulmonol 47, 682-691 (2012)). Interestingly, though bradycardia has been implicated as an explanation of sudden death among FD patients (Bernardi, L., et al. Am J Respir Crit Care Med 167, 141-149 (2003); Brown, C. M., et al. Clin Sci (Lond) 104, 163-169 (2003); Rotstein, A., Charrow, J. & Deal, B. J. Pediatr Cardiol 29, 202-204 (2008); Solaimanzadeh, I., et al. Auton Neurosci 144, 76-82 (2008)), which could be a consequence of a hyperactive parasympathetic tone. Adding to the controversy, FD patients also display pupillary hypersensitivity to parasympathomimetic treatments (Bar-Aluma, B. E. Familial Dysautonomia. in GeneReviews((R)) (eds. Adam, M. P., et al.) (Seattle (WA), 1993)), and parasympathetic hypersensitivity induced lung stretching is also proposed as a reason of the breath-holding spells in FD (Maayan, C., Katz, E., Begin, M., Yuvchev, I. & Kharasch, V. S. Clin Pediatr (Phila) 54, 174-178 (2015)). Here, the behavior between healthy and FD hPSC-parasymNs was compared and it was found that FD parasymNs are hyperactive. Though this phenotype is subtler than the FD symN phenotype in vitro (FIG. 7A-7D). It has also been shown the impaired crosstalk within the FD ANS (FIG. 8C-8G), which may explain the instability and unpredictability of FD symptoms. Together with the success in establishing co-culture models using hPSC-parasymNs (FIG. 5G), this system provides a platform of human parasymNs to study parasympathetic neuropathology in FD.

The interaction between the RAAS and the SNS has been extensively studied (Huang, B. S. & Leenen, F. H. Curr Heart Fail Rep 6, 81-88 (2009); Cody, R. J. Am J Cardiol 80, 9J-14J (1997)). The understanding of the regulatory role of RAAS on PSNS, in contrast, needs further works, despite the studies showing that RAAS may interfere with parasympathetic drive and baroreflex through Ang II. Here, it has been showed that these hPSC-parasymNs exhibit functional reactivity to Ang II level changes (FIGS. 9B-9C). Also shown is the anti-inflammatory activity of hPSC-parasymNs in a model that simulates heart damage in COVID-19 (FIGS. 9D-9G), a well-known COVID-19 complication that acts through the RAAS system. The current in vitro model therefore can be applied to study parasymN responsiveness to treatments and manipulations of other RAAS components, such as Ang 1-7 and MasR. The results could then be used for mechanistic studies and compound screenings to activate PSNS in COVID-19 patients as a therapeutic strategy in the future.

Autoantibodies against type 3 MusR have been proposed as a reason for ANS dysfunction in Sjogren's syndrome (Li, J., et al. Lab Invest 84, 1430-1438 (2004)). Here, it has been shown that such antibodies not just target the downstream tissues, they directly recognize and hypoactivate presynaptic parasymNs (FIGS. 10A-10G). Excessive extracellular ACh was also identified, caused by reduced AChE activity when cells were treated with Sjogren's syndrome patient IgGs, possibly suggesting an intrinsic parasympathetic response to inflammation (FIGS. 10D-10E) (Shaked, I., et al. Immunity 31, 965-973 (2009)). It is also possible that the reduced parasymN activity in response to Sjogren's syndrome patient IgGs, is a result of cellular toxicity from excessive ACh taken up via the MusRs (FIG. 10G).

Dual innervation of the ANS is commonly found in most tissues in the body, but to date it remains unclear whether adipose tissues have parasympathetic innervation in addition to sympathetic innervation. In 2002, Kreier et al. reported that the WAT is innervated by parasymNs, detected using the retrograde pseudorabies virus (PRV) tracing technique in the rat model. Later in 2006, Giordano et al., interestingly, reported the opposite result using PRV and immunohistochemistry in hamsters (Giordano, A., et al. Am J Physiol Regul Integr Comp Physiol 291, R1243-1255 (2006)). Such difference among species prompted us to test WAT innervation using these human-based parasymN model. The results showed that hPSC-parasymNs can innervate and regulate the maturity as well as functionality of 3T3-L1-derived white adipocytes (FIGS. 11A-11J), although there are some limits to the interpretation of these results due to its in vitro nature. Although the 3T3-L1-derived adipocyte model is one of the most widely used in vitro models of adipose tissue due to its stability and efficiency of differentiation (Green, H. & Meuth, M. Cell 3, 127-133 (1974)), it would be worthy in the future to establish an all-human co-culture model using human preadipocyte cell lines or hPSC-derived adipocytes.

In sum, a parasymN differentiation platform has been described and the versatility and utility of this model has been demonstrated by employing it to model various human ailments. The studies open new avenues to study the human PSNS, which is equally important to the SNS, yet is vastly understudied. With this new tool, the responses and interactions of the PSNS and the SNS can be thoroughly compared, and the imbalance of autonomic efference can be modeled to accelerate the discovery of new therapeutics for a large variety of human diseases.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A method of making parasympathetic neurons (parasymN) comprising culturing of Schwann Cell Progenitors (SCPs) in chemically-defined parasymN differentiation media.

2. The method of claim 1, wherein the chemically-defined parasymN differentiation media comprises one or more of GDNF, BDNF, CNTF, and FBS, optionally all of GDNF, BDNF, CNTF, and FBS, and optionally free from NGF.

3. The method of claim 2, wherein chemically-defined parasymN differentiation media comprises B27, L-Glutamine, FBS, GDNF, BDNF, CNTF, ascorbic acid, dbcAMP, and retinoic acid, optionally wherein the media is Neurobasal medium further comprising B27, L-Glutamine, 1% FBS, 25 ng/ml GDNF, 25 ng/ml BDNF, 25 ng/ml CNTF, 200 μM ascorbic acid, 0.2 mM dbcAMP and 0.125 μM retinoic acid, or a variation thereof with 20%, 10%, or 5%, more or less of the foregoing ingredients.

4. The method of claim 2 comprising culturing the cells for about two or more weeks.

5. The method of claim 4, wherein the SCPs are prepared by culturing Neural Crest cells (NCC) in a chemically-defined SCP differentiation media.

6. The method of claim 5, wherein the SCPs are cultured as spheroids.

7. The method of claim 6, wherein the SCPs are cultured in SCP differentiation media for about 6-14 days.

8. The method of claim 5, wherein the SCP differentiation media comprises
(i) B27, L-Glutamine, FGF2, dbcAMP, and NRG1, optionally wherein the media is Neurobasal medium further comprising B27, L-Glutamine, 10 ng/ml FGF2, 10011M dbcAMP, and 20 ng/ml NRG1, or a variation thereof with 20%, 10%, or 5%, more or less of the foregoing ingredients; or
(ii) N2, B27, BSA, GlutaMAX, (β-mercaptoethanol, CT 99021, SB431542, and NRG1, optionally wherein the media is DMEM/F12 and Neurobasal medium (1:1 mix) further comprising 1× N2, 1× B27, 0.005% BSA, 2 mM GlutaMAX, 0.11 mM (β-mercaptoethanol, 3 μM CT 99021, and 20 μM SB431542, and wherein 50 ng/mL NRG1 is only added after 6 days, or a variation thereof with 20%, 10%, or 5%, more or less of the foregoing ingredients.

9. The method of claim 6 where the cells are cultured on a substrate coated with Polyornithine (PO)/laminin (LM)/fibronectin (FN).

10. The method of claim 5, wherein the NCC are prepared by culturing stem cells, optionally embryonic stems cells or induced pluripotent stem cells, in a chemically-defined NCC induction media.

11. The method of claim 10, wherein the NCC induction media comprises a transforming growth factor beta (TGF(3)/Activin-Nodal signaling inhibitor and an activator wingless (Wnt) signaling.

12. The method of claim 11, wherein the NCC induction media comprises BMP4, SB431542, and CHIR99021, optionally wherein the induction media is Essential 6 medium further comprising 0.4 ng/ml BMP4, 1011M SB431542 and 300 nM CHIR99021 for days 0-1, and Essential 6 medium further comprising 1011M SB431542 and 0.7511M CHIR99021 for days 2 on, or a variation thereof with 20%, 10%, or 5%, more or less of the foregoing ingredients.

13. The method of claim 12, wherein the NCC are cultured in NCC induction media for 10-12 days.

14. The method of claim 1, wherein the method induces differentiation of about 50% of the cells into parasymN.

15. The method of claim 1, wherein the parasymN are characterized by one or more of a neuron-like cell morphology, the appearance of well-developed neurite bundles, expression of one or more autonomic markers such as ASCL1, PHOX2B, PRPH, and/or CHRNA3, one or more parasympathetic markers such as ChAT, VAChT, ChT, and/or NPY2R, reduced expression of SOX/0 e.g., relative to SCPs and/or SCs, predominate expression of ChAT relative TH, e.g., compared to symNs, HOX 1-5 positive, optionally except HOX 2, one or more cholinergic muscarinic receptors (MusR), preferably at least M2 and/or M4, functionally activated by bethanechol (BeCh), functionally inactivated by atropine, insensitivity to 6-OHDA, and ability to build functional connectivity to and/or innervate target,
optionally wherein the parasymN comprise PRPH neurons, optionally wherein 70%, 75%, 80%, 85%, 90%, 95%, or more of PRPH neurons express ChAT.

16. The method of claim 15, further comprising enriching the parasymN, optionally comprising FACS and/or immunopanning.

17. The method of 15 comprising isolating parasymN from other cells in the culture, optionally wherein the other cells comprise one or more of stem cells, NCC, SCP, and SC.

18. A population of cells formed according to the method of claim 1.

19. Conditioned media formed by the population of cells according to claim 18.

20. Induced parasymN cells comprising one or more of a neuron-like cell morphology, the appearance of well-developed neurite bundles, expression of one or more autonomic markers optionally ASCL1, PHOX2B, PRPH, and/or CHRNA3, one or more parasympathetic markers such as ChAT, VAChT, ChT, and/or NPY2R, reduced expression of SOX/0 relative to SCPs and/or SCs, predominate expression of ChAT relative TH compared to symNs, HOX 1-5 positive, optionally except HOX 2, one or more cholinergic muscarinic receptors (MusR), optionally at least M2 and/or M4, functionally activated by bethanechol (BeCh), functionally inactivated by atropine, insensitivity to 6-OHDA, and ability to build functional connectivity to and/or innervate target tissues.


SEQUENCE LISTING (66 sequences)

SEQ ID NO: 1 - DNA, 23 bp, synthetic construct
  source 1..23: mol_type = other DNA
cagcgtcagtttcaacctgtacg

SEQ ID NO: 2 - DNA, 22 bp, synthetic construct
  source 1..22: mol_type = other DNA
gcaggtgactttggctacaagc

SEQ ID NO: 3 - DNA, 23 bp, synthetic construct
  source 1..23: mol_type = other DNA
ccatgttctgaccttcctggatg

SEQ ID NO: 4 - DNA, 22 bp, synthetic construct
  source 1..22: mol_type = other DNA
cggattaacgcagctgttggtg

SEQ ID NO: 5 - DNA, 22 bp, synthetic construct
  source 1..22: mol_type = other DNA
tctcatcctactcgtcggacga

SEQ ID NO: 6 - DNA, 23 bp, synthetic construct
  source 1..23: mol_type = other DNA
ctgcttccaaagtccattcgcac

SEQ ID NO: 7 - DNA, 22 bp, synthetic construct
  source 1..22: mol_type = other DNA
catcacctggaggacttctacc

SEQ ID NO: 8 - DNA, 23 bp, synthetic construct
  source 1..23: mol_type = other DNA
cagtgtactggatgctcttcagg

SEQ ID NO: 9 - DNA, 20 bp, synthetic construct
  source 1..20: mol_type = other DNA
gccttctacaggctccatcg

SEQ ID NO: 10 - DNA, 20 bp, synthetic construct
  source 1..20: mol_type = other DNA
gctctcacaaaagccagtgc

SEQ ID NO: 11 - DNA, 22 bp, synthetic construct
  source 1..22: mol_type = other DNA
ctggctggtttcctttgtgctc

SEQ ID NO: 12 - DNA, 22 bp, synthetic construct
  source 1..22: mol_type = other DNA
ggagaggaactggatgtagcac

SEQ ID NO: 13 - DNA, 22 bp, synthetic construct
  source 1..22: mol_type = other DNA
tgctgtcacctttggtacggct

SEQ ID NO: 14 - DNA, 22 bp, synthetic construct
  source 1..22: mol_type = other DNA
tggttggcaacaggctccttct

SEQ ID NO: 15 - DNA, 22 bp, synthetic construct
  source 1..22: mol_type = other DNA
acgagagccatctactccatcg

SEQ ID NO: 16 - DNA, 22 bp, synthetic construct
  source 1..22: mol_type = other DNA
tgtcggctttcctctccaagtc

SEQ ID NO: 17 - DNA, 23 bp, synthetic construct
  source 1..23: mol_type = other DNA
ctcccatgaacctctacaccgtg

SEQ ID NO: 18 - DNA, 22 bp, synthetic construct
  source 1..22: mol_type = other DNA
gacgcagaagtagcggtcaaag

SEQ ID NO: 19 - DNA, 22 bp, synthetic construct
  source 1..22: mol_type = other DNA
ccaccatcacttttggcactgc

SEQ ID NO: 20 - DNA, 22 bp, synthetic construct
  source 1..22: mol_type = other DNA
aggtccttggttcgcttctctg

SEQ ID NO: 21 - DNA, 22 bp, synthetic construct
  source 1..22: mol_type = other DNA
tggagaccaacctgtggctcaa

SEQ ID NO: 22 - DNA, 22 bp, synthetic construct
  source 1..22: mol_type = other DNA
cagcacaatgtctggcttccag

SEQ ID NO: 23 - DNA, 22 bp, synthetic construct
  source 1..22: mol_type = other DNA
atctggttgcctgacatcgtgc

SEQ ID NO: 24 - DNA, 22 bp, synthetic construct
  source 1..22: mol_type = other DNA
ttgcaggcgctcttgtagatgg

SEQ ID NO: 25 - DNA, 22 bp, synthetic construct
  source 1..22: mol_type = other DNA
aaaagccgtggctgggaactgt

SEQ ID NO: 26 - DNA, 22 bp, synthetic construct
  source 1..22: mol_type = other DNA
gcacttgagcataggtggctga

SEQ ID NO: 27 - DNA, 22 bp, synthetic construct
  source 1..22: mol_type = other DNA
gccttcatcctcactggctact

SEQ ID NO: 28 - DNA, 22 bp, synthetic construct
  source 1..22: mol_type = other DNA
cagcactgtgaccacctttctc

SEQ ID NO: 29 - DNA, 22 bp, synthetic construct
  source 1..22: mol_type = other DNA
gtctcctctgacttcaacagcg

SEQ ID NO: 30 - DNA, 22 bp, synthetic construct
  source 1..22: mol_type = other DNA
accaccctgttgctgtagccaa

SEQ ID NO: 31 - DNA, 22 bp, synthetic construct
  source 1..22: mol_type = other DNA
gagcagccaagctgaagagaac

SEQ ID NO: 32 - DNA, 24 bp, synthetic construct
  source 1..24: mol_type = other DNA
gccatttcttagagttcaggcatg

SEQ ID NO: 33 - DNA, 20 bp, synthetic construct
  source 1..20: mol_type = other DNA
caatccgccacgtctccttc

SEQ ID NO: 34 - DNA, 17 bp, synthetic construct
  source 1..17: mol_type = other DNA
ccaggccatctgcaggc

SEQ ID NO: 35 - DNA, 20 bp, synthetic construct
  source 1..20: mol_type = other DNA
cgaaatagacgaggccagcg

SEQ ID NO: 36 - DNA, 19 bp, synthetic construct
  source 1..19: mol_type = other DNA
cggcccggtcatatcatgg

SEQ ID NO: 37 - DNA, 20 bp, synthetic construct
  source 1..20: mol_type = other DNA
cgccctttgagcagaacctc

SEQ ID NO: 38 - DNA, 21 bp, synthetic construct
  source 1..21: mol_type = other DNA
cgtttgcggtcagttcctgag

SEQ ID NO: 39 - DNA, 19 bp, synthetic construct
  source 1..19: mol_type = other DNA
gcagcaagcacaaagagga

SEQ ID NO: 40 - DNA, 21 bp, synthetic construct
  source 1..21: mol_type = other DNA
cgtctggtacttggtgtaggg

SEQ ID NO: 41 - DNA, 17 bp, synthetic construct
  source 1..17: mol_type = other DNA
gcgagccctcgattcgc

SEQ ID NO: 42 - DNA, 23 bp, synthetic construct
  source 1..23: mol_type = other DNA
gaattgcccagggaatccttctc

SEQ ID NO: 43 - DNA, 20 bp, synthetic construct
  source 1..20: mol_type = other DNA
ccacggtcaactcgggacct

SEQ ID NO: 44 - DNA, 20 bp, synthetic construct
  source 1..20: mol_type = other DNA
ttcctacagaccccgccgtg

SEQ ID NO: 45 - DNA, 22 bp, synthetic construct
  source 1..22: mol_type = other DNA
aggctgtagcagtcctgaaagg

SEQ ID NO: 46 - DNA, 22 bp, synthetic construct
  source 1..22: mol_type = other DNA
cttcctccactgtgacagtctg

SEQ ID NO: 47 - DNA, 22 bp, synthetic construct
  source 1..22: mol_type = other DNA
ctatcctggctgtgctgctctt

SEQ ID NO: 48 - DNA, 22 bp, synthetic construct
  source 1..22: mol_type = other DNA
actcactggaccagaaggagca

SEQ ID NO: 49 - DNA, 22 bp, synthetic construct
  source 1..22: mol_type = other DNA
actcctagaggtgaactggtcc

SEQ ID NO: 50 - DNA, 22 bp, synthetic construct
  source 1..22: mol_type = other DNA
catggatcaccaaggagttgcc

SEQ ID NO: 51 - DNA, 23 bp, synthetic construct
  source 1..23: mol_type = other DNA
ggctttcaaccatctcattcccg

SEQ ID NO: 52 - DNA, 22 bp, synthetic construct
  source 1..22: mol_type = other DNA
gttgaggtctgtgaacggtgct

SEQ ID NO: 53 - DNA, 21 bp, synthetic construct
  source 1..21: mol_type = other DNA
ctaccccgacatctacactcg

SEQ ID NO: 54 - DNA, 19 bp, synthetic construct
  source 1..19: mol_type = other DNA
ctcctgcttgcgaaacttg

SEQ ID NO: 55 - DNA, 20 bp, synthetic construct
  source 1..20: mol_type = other DNA
gtgcccgtccattcttttgc

SEQ ID NO: 56 - DNA, 20 bp, synthetic construct
  source 1..20: mol_type = other DNA
gtcaccacctccccattccg

SEQ ID NO: 57 - DNA, 19 bp, synthetic construct
  source 1..19: mol_type = other DNA
ccaggcccactacaagagc

SEQ ID NO: 58 - DNA, 18 bp, synthetic construct
  source 1..18: mol_type = other DNA
ctctggcctgaggggtgc

SEQ ID NO: 59 - DNA, 23 bp, synthetic construct
  source 1..23: mol_type = other DNA
gaagaaatccgaactgaaggagc

SEQ ID NO: 60 - DNA, 22 bp, synthetic construct
  source 1..22: mol_type = other DNA
tcctggaagtcacattcgccgt

SEQ ID NO: 61 - DNA, 22 bp, synthetic construct
  source 1..22: mol_type = other DNA
tcggctttgtgaaggtgctgca

SEQ ID NO: 62 - DNA, 22 bp, synthetic construct
  source 1..22: mol_type = other DNA
tcactctcggtcttgttggcac

SEQ ID NO: 63 - DNA, 22 bp, synthetic construct
  source 1..22: mol_type = other DNA
gctggacaagtgtcatcacctg

SEQ ID NO: 64 - DNA, 22 bp, synthetic construct
  source 1..22: mol_type = other DNA
cctgtactggaaggcgatctca

SEQ ID NO: 65 - DNA, 23 bp, synthetic construct
  source 1..23: mol_type = other DNA
gctgtttgcttccaaggctatcc

SEQ ID NO: 66 - DNA, 22 bp, synthetic construct
  source 1..22: mol_type = other DNA
gaaggcgaacaggactgtagag